SEQUENCE LISTING (16 sequences)

SEQ ID NO: 1 - protein, 13 aa, Homo sapiens
LVYKDPARPKIQK

SEQ ID NO: 2 - protein, 13 aa, Mus musculus
LVYKDPARPNTQK

SEQ ID NO: 3 - protein, 116 aa, synthetic construct
DVHLQESGPGLVKPSQSLSLTCTVTGFSITSDYAWNWIRQFPGNKLEWMGSIFSSGSINYNPSLKSRISITRDTSRNQFFLQLNSVTTADAGTYYCARGGTGTDYWGQGTTLTVSS

SEQ ID NO: 4 - protein, 107 aa, synthetic construct
DIQMTQTTSSLSASLGDRVTISCRASQDISNYLSWYQQKPDGTIKLLIYYTSRLHSGVSSRFSGSGSGTDYSLTISNLEQEDFATYFCQQGHTLPPTFGGGTKLEIK

SEQ ID NO: 5 - protein, 6 aa, synthetic construct
SDYAWN

SEQ ID NO: 6 - protein, 16 aa, synthetic construct
SIFSSGSINYNPSLKS

SEQ ID NO: 7 - protein, 7 aa, synthetic construct
GGTGTDY

SEQ ID NO: 8 - protein, 11 aa, synthetic construct
RASQDISNYLS

SEQ ID NO: 9 - protein, 7 aa, synthetic construct
YTSRLHS

SEQ ID NO: 10 - protein, 9 aa, synthetic construct
QQGHTLPPT

SEQ ID NO: 11 - protein, 116 aa, synthetic construct
QVQLQESGPGLVKPSQTLSLTCTVSGFSITSDYAWNWIRQPPGKGLEWIGSIFSSGSINYNPSLKSRVTISRDTSKNQFSLKLSSVTAADTAVYYCARGGTGTDYWGQGTLVTVSS

SEQ ID NO: 12 - protein, 107 aa, synthetic construct
DIQMTQSPSSLSASVGDRVTITCRASQDISNYLSWYQQKPGKAIKLLIYYTSRLHSGVPSRFSGSGSGTDYTLTISSLQPEDFATYFCQQGHTLPPTFGGGTKVEIK

SEQ ID NO: 13 - protein, 116 aa, synthetic construct
QVQLQESGPGLVKPSQTLSLTCTVSGFSITSDYAWNWIRQPPGKGLEWIGSIFSSGSINYNPSLKSRVTISRDTSKNQFSLKLSSVTAADTGVYYCARGGTGTDYWGQGTLVTVSS

SEQ ID NO: 14 - protein, 107 aa, synthetic construct
DIQMTQSPSSLSASVGDRVTITCRASQDISNYLSWYQQKPGGAPKLLIYYTSRLHSGVPSRFSGSGSGTDYTLTISSLQPEDFATYFCQQGHTLPPTFGGGTKVEIK

SEQ ID NO: 15 - protein, 116 aa, synthetic construct
QVQLQESGPGLVKPSQTLSLTCTVSGFSITSDYAWNWIRQPPGKGLEWMGSIFSSGSINYNPSLKSRITISRDTSKNQFSLKLSSVTAADTGVYYCARGGTGTDYWGQGTLVTVSS

SEQ ID NO: 16 - protein, 107 aa, synthetic construct; identical to SEQ ID NO: 14

(19) United States
(12) Patent Application Publication
(10) Pub. No.: US 2025/0243269 A1
(43) Pub. Date: Jul. 31, 2025

(54) ANTI-FSH ANTIBODIES FOR NEURODEGENERATIVE DISEASES

(71) Applicant: ICAHN SCHOOL OF MEDICINE AT MOUNT SINAI, New York, NY (US)

(72) Inventor: Mone Zaidi, New York, NY (US)

(21) Appl. No.: 18/290,611

(22) PCT Filed: Jul. 21, 2022
(86) PCT No.: PCT/US2022/037871

§ 371 Date: Jan. 19, 2024

Related U.S. Application Data
(60) Provisional application No. 63/224,067, filed on Jul. 21, 2021.

(51) Int. Cl. C07K 16/26; A61K 39/00; A61P 25/28

(52) U.S. Cl. CPC C07K 16/26; A61P 25/28; A61K 2039/505; C07K 2317/24; C07K 2317/76

(57) ABSTRACT

The present disclosure provides compositions and methods for treating neurodegenerative diseases, in particular, Alzheimer's Disease, by using anti-FSH antibodies in a subject in need thereof. In some embodiments, the subject has a condition in which FSH levels are elevated. The methods include administering to said subject a therapeutically effective amount of an anti-FSH antibody or an antigen-binding portion thereof.

### CROSS-REFERENCE TO RELATED APPLICATIONS

This application is a U.S. National Phase application under 35 U.S.C. § 371 of International Patent Application No. PCT/US2022/037871 filed Jul. 21, 2022, which claims priority to U.S. provisional application No. 62/982,521 filed Feb. 27, 2020. The contents of these applications are incorporated herein by reference in their entirety.

### STATEMENT REGARDING FEDERALLY SPONSORED RESEARCH

This invention was made with government support under U19 AG60917 and U19 AG60917-02S1 awarded by the National Institutes of Health/National Institute on Aging. The government has certain rights in the invention.

### SEQUENCE LISTING

This application contains a Sequence Listing that has been submitted electronically as an XML file named 27527-0183US1_SL_ST26.xml. The XML file, created on Dec. 12, 2024, is 15,750 bytes in size. The material in the XML file is hereby incorporated by reference in its entirety.

### TECHNICAL FIELD

This disclosure relates generally to methods of treating neurodegenerative diseases in a subject with anti-FSH antibodies. In particular, the antibodies of the disclosure can be used to treat all forms of Alzheimer's Disease (AD).

### BACKGROUND

Neurodegenerative diseases of the aging population such as Alzheimer's Disease (AD) affect about 35 million people worldwide, with an estimated 7 million new cases every year (World Health Organization; Dementia Fact Sheet; September 2020). AD and other neurodegenerative disorders pose a major global health problem resulting in progressive dementia, profound disability, and impaired quality of life. Constituting ˜70% of the AD population (Andersen, K. et al. Neurology 53, 1992-1997, (1999)), women have a greater life-time risk for AD than men, and display a ˜3-fold higher rate of disease progression (Laws, K. R., et al. World J Psychiatry 6, 54-65, (2016)) with a broader spectrum of behavioral symptoms (Koran, M. E. I., et al. Brain Imaging Behav 11, 205-213, (2017); Marongiu, R. Front Aging Neurosci 11, 242, (2019)).

Yet, there are few treatment options for Alzheimer's Disease and other neurodegenerative diseases regardless of gender, with current medications unable to stop the damage to neurons, and limited symptomatic-only treatments available. Thus, there is an urgent and wide-ranging need for treatments of neurodegenerative diseases, such as Alzheimer's Disease, which are associated with beta-amyloid deposits and/or neurofibrillary tangles.

### SUMMARY

This disclosure is based, at least in part, on the finding that anti-FSH antibodies can improve cognition in mice with Alzheimer's Disease.

Disclosed herein, is a method for treating Alzheimer's Disease (AD), preventing the onset of AD, reducing cognitive or functional decline, or reducing symptom load in AD a subject in need or at risk thereof, comprising administering to said subject a therapeutically effective amount of an anti-Follicle Stimulating Hormone (FSH) antibody or an antigen-binding portion thereof, wherein the anti-FSH antibody, or antigen-binding portion thereof comprises (a) a heavy chain variable sequence comprising a sequence that is at least 90% identical to a sequence selected from SEQ ID NOs: 3, 11, 13, and 15; (b) a light chain variable sequence comprising a sequence that is at least 90% identical to a sequence selected from SEQ ID NOs: 4, 12, 14, and 17; (c) a heavy chain CDR1 (CDRH1) comprising SEQ ID NO:5; (d) a heavy chain CDR2 (CDRH2) comprising SEQ ID NO:6; (e) a heavy chain CDR3 (CDRH3) comprising SEQ ID NO: 7; (f) a light chain CDR1 (CDRL1) comprising SEQ ID NO:8; (g) a light chain CDR2 (CDRL2) comprising SEQ ID NO:9; and (h) a light chain CDR3 (CDRL3) comprising SEQ ID NO:10.

In some embodiments, the subject has a condition in which FSH levels are elevated. In some embodiments, the subject is female. In some embodiments, subject is perimenopausal or postmenopausal. In some embodiments, the subject is male.

In some embodiments, the condition is a genetic disease, chemotherapy, surgical menopause, or orchiectomy. In some embodiments, the genetic disease is Turners syndrome.

In some embodiments, the method alters one or more of the following in the subject in need thereof: (a) reduces Aβ accumulation; (b) reduces amyloid plaques; (c) reduces Tau accumulation in the brain; and (d) enhances cognitive function. In some embodiments, the one or more of Aβ accumulation, amyloid plaques, and Tau accumulation in the brain is lower by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the subject or in a control.

In some embodiments, the cognitive function is enhanced by at least about 20%, at least about 30%, at least about 40%, or at least about 50%, at least about 60%, at least about 70%, at least about 80%, or at least about 90%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3-alternative forced choice task; and the Benton Visual Retention Test.

In some embodiments, the subject is concurrently treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of NGF, CERE-110, beta-blocker, human amyloid vaccine, beta or gamma secretase inhibitor, nicotinic or muscarinic agonist, and a second antibody.

In some embodiments, the cholinesterase inhibitor is selected from the group consisting of galantamine, rivastigmine, tacrine, and donepezil. In some embodiments, the NMDA receptor antagonist is selected from the group consisting of ketamine, methadone, memantine, amantadine, and dextromethorphan or a salt thereof. In some embodiments, the antipsychotic agent is selected from the group consisting of aripiprazole, risperidone, olanzapine, quetiapine, or haloperidol. In some embodiments, the benzodiazepine is selected from the group consisting of lorazepam, oxazepam and temazepam. In some embodiments, the tricyclic antidepressant is nortriptyline. In some embodiments, the agent is a hormone selected from the group consisting of estrogen, progesterone and leuprolide. In some embodiments, the agent is a vitamin selected from the group consisting of folate and nicotinamide. In some embodiments, the second antibody is selected from the group consisting of bapineuzumab, solanezumab, gantenerumab, crenezumab, ponezumab, BAN2401, and aducanumab.

In some embodiments, the anti-FSH antibody or antigen-binding portion thereof is administered subcutaneously, intramuscularly, intravenously, intrathecally, or intracranially to the subject. In some embodiments, the anti-FSH antibody or antigen-binding portion thereof is administered to a subject in need thereof at a dose of about 0.2 to 50 mg/kg of the subject's body weight. In some embodiments, the anti-FSH antibody or antigen-binding portion thereof is administered to a subject in need thereof twice a week, every week, every 2 weeks, every month, every two months, or every six months.

In some embodiments, the cognitive decline is assessed by determining the subject's score before and after administration of said anti-FSH antibody or antigen-binding fragment thereof, using an Alzheimer's Disease Assessment Scale-Cognition (ADAS-Cog) test.

In some embodiments, the reduction in cognitive decline as measured by ADAS-Cog is at least 10%, at least 15%, at least 20%, at least 25%, at least 30%, at least 35%, at least 40%, or at least 45% relative to a placebo.

In some embodiments, the subject has mild, moderate, or severe AD. In some embodiments, the treatment is prophylactic for completely or partially preventing AD or symptoms thereof in the subject. In some embodiments, the treatment is therapeutic for partially or completely curing AD or symptoms associated with AD in the subject.

Also disclosed herein is a pharmaceutical composition comprising an isolated anti-Follicle Stimulating Hormone (FSH) antibody, or antigen-binding portion thereof, and a pharmaceutically acceptable carrier or excipient, wherein the composition is capable of crossing the blood brain barrier from the blood into the brain, wherein the anti-FSH antibody or antigen-binding portion thereof comprises (a) a heavy chain variable sequence comprising a sequence that is at least 90% identical to a sequence selected from SEQ ID NOs: 3, 11, 13, and 15; (b) a light chain variable sequence comprising a sequence that is at least 90% identical to a sequence selected from SEQ ID NOs: 4, 12, 14, and 17; (c) a heavy chain CDR1 (CDRH1) comprising SEQ ID NO:5; (d) a heavy chain CDR2 (CDRH2) comprising SEQ ID NO:6; (e) a heavy chain CDR3 (CDRH3) comprising SEQ ID NO:7; (f) a light chain CDR1 (CDRL1) comprising SEQ ID NO:8; (g) a light chain CDR2 (CDRL2) comprising SEQ ID NO:9; and (h) a light chain CDR3 (CDRL3) comprising SEQ ID NO:10.

In some embodiments, the composition disclosed herein is use in treating Alzheimer's Disease (AD), preventing the onset of AD, or reducing cognitive or functional decline in AD a subject in need or at risk thereof, wherein the composition is administered intravenously, intrathecally, or intracranially to the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows the novel/familiar ratio in a Novel Object Recognition Test for mice receiving goat IgG (control) or Hu6 (test antibody) (200 μg/mouse, daily, i.p. for 4 weeks).

FIG. 1B shows freezing time (seconds) in a Freezing Conditioning Test for mice receiving IgG (control) or Hu6 (test antibody). Unpaired Student's t-test; n=3-4 per group; *p<0.05

FIG. 2A shows the effect of monoclonal anti-FSH antibody (Hu6) on cognition in AD mice. The novel/familiar ratio in a Novel Object Recognition Test is shown for wildtype (WT) or APP/PS1 mice receiving human IgG (control; white bar) or Hu6 (test antibody; black bar); 100 μg/mouse, for 4 months; 5 days a week for the first two months, and 3 days a weeks for the next two months.

FIG. 2B shows the effect of monoclonal anti-FSH antibody (Hu6) on cognition in AD mice. % freezing time in a Freezing Conditioning Test is shown for WT or APP/PS1 mice receiving IgG (control) or Hu6 (test antibody) as indicated. FIG. 2C depicts % freezing time for wildtype (WT) or APP/PS1 mice receiving IgG (white bar) or Hu6 (black bar) in the 1^{st} to 3^{rd} minute, 4^{th} to 7^{th} minute and 8^{th} to 10^{th} minute. Unpaired Student's t-test; n=3-4 per group; *p<0.05

FIG. 3 shows the permeation of the fluorescent-labeled anti-FSH antibody, Hu6, into the mouse brain.

FIG. 4A shows interactions between the anti-FSH antibody and the FSH receptor as determined by epitope fine mapping.

FIG. 4B shows interactions between the anti-FSH antibody and the FSH receptor as determined by epitope fine mapping.

FIG. 5A shows the biodistribution and excretion of anti-FSH antibodies in a representative Cynomolgus monkey, with whole body PET/CT image at 48 hours and 120 hours following a single i.v. injection of ⁸⁹Zr-MS-Hu6 (1.3 mg, ˜1.3 mCi).

FIG. 5B shows a plot of quantitation (SUVs) of multiple organs and serum radioactivity (y-counts) following a single i.v. injection of ⁸⁹Zr-MS-Hu6 (1.3 mg, ˜1.3 mCi).

FIG. 6A shows plots of physiological parameters (monitored up to 100 minutes after injecting ⁸⁹Zr-MS-Hu6 as a single i.v. bolus dose (1.3 mg, ˜1.3 mCi) into the tail veins of Cynomolgus monkeys.

FIG. 6B shows plots of serum biochemistry (at days 0, 2 and 5) after injecting ⁸⁹Zr-MS-Hu6 as a single i.v. bolus dose (1.3 mg, ˜1.3 mCi) into the tail veins of Cynomolgus monkeys.

FIG. 7A shows comparisons of twelve physicochemical parameters that were computationally-derived using the Protein-Sol program for anti-FSH antibodies against experimental derivations of 48 FDA-approved antibodies and 89 antibodies in late-stage clinical development.

FIG. 7B shows a table summarizing results of twelve physicochemical parameters that were computationally-derived using the Protein-Sol program for anti-FSH antibodies and a computational variation of the anti-FSH antibody, in which the CDR region was scrambled.

FIG. 8A shows the results of isoelectric focusing to determine the isoelectric point (pI) for anti-FSH antibodies.

FIG. 8B shows a plot of results of thermal shift assays that were used to evaluate stability of both Fc and Fab regions of anti-FSH antibody MS-Hu6 in formulation versus anti-FSH antibody MS-Hu6 in PBS.

FIG. 8C shows a plot of results of thermal shift assays that were used to evaluate stability of FSH binding to the Fab region of anti-FSH antibody MS-Hu6 in formulation.

FIG. 8D shows a plot of UV absorbance (280 nm) readout of self-interaction chromatography (SIC) to assess binding of anti-FSH antibody MS-Hu6 in formulation versus anti-FSH antibody MS-Hu6 in PBS with self or human IgG, respectively.

FIG. 8E shows shows a plot of UV absorbance (280 nm) readout of cross-interaction chromatography (CIC) to assess binding of anti-FSH antibody MS-Hu6 in formulation versus anti-FSH antibody MS-Hu6 in PBS with self or human IgG, respectively.

FIG. 8F shows a plot of results of hydrophobic chromatography showing UV absorbance (280 nm) of the eluate from a butyl sepharose column upon passing anti-FSH antibody MS-Hu6 at pH 6.5 over 20 minutes at a flow rate of 1 mL/min (retention time shown).

FIG. 8G shows representative size exclusion chromatograms and area under the peak for anti-FSH antibody MS-Hu6 in PBS or anti-FSH antibody MS-Hu6 in formulation following stress testing by 3 cycles of freeze-thaw or incubation at 4° C., 37° C. and 50° C. for 1 week.

Like reference symbols in the various drawings indicate like elements.

### DETAILED DESCRIPTION

This disclosure is based, in part, on the unexpected discovery that anti-FSH antibodies can improve cognition in mice with Alzheimer's Disease.

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present application will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

The nomenclatures used in connection with, and the laboratory procedures and techniques of biochemistry, immunology, microbiology, molecular biology, and virology described herein are those well-known and commonly used in the art.

Throughout this specification and embodiments, the word comprise, or variations such as comprises or comprising, will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a,” “an,” and “the” are used herein to refer to one or to more than one (i.e., to at least one) of the grammatical object of the article. By way of example, an element means one element or more than one element. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of +10% within the range of the significant digit.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the Markush group or other grouping of alternatives.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

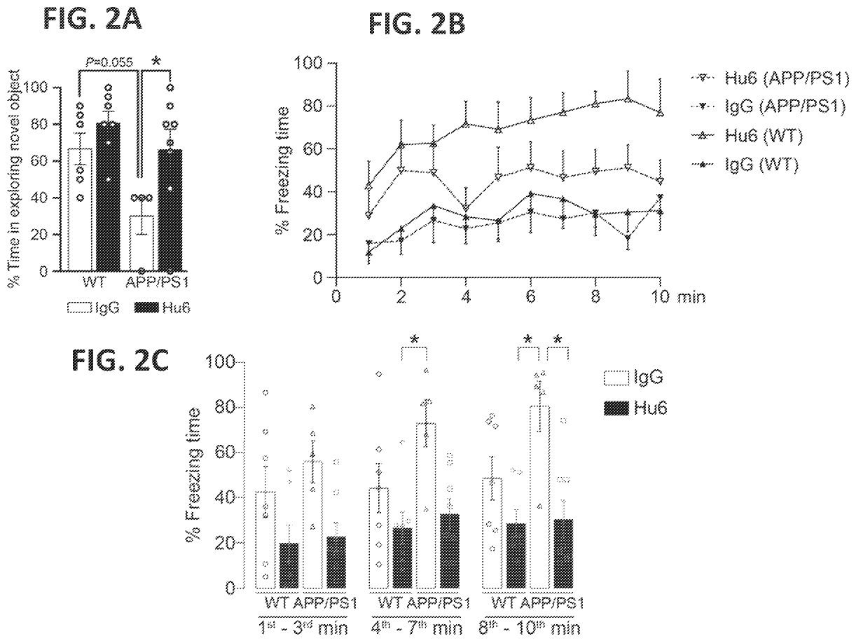

As used herein, the term “neurodegenerative diseases” or “neurodegenerative disorders” refers to a diseases characterized by the deposition of insoluble protein inside and outside brain cells and/or cells of the neuromuscular system. In some embodiments, the disorder is Alzheimer's Disease. Other diseases include but are not limited to progressive supranuclear palsy, frontotemporal lobar degeneration (Pick's disease), corticobasal degeneration and post-encephalitic parkinsonism, frontotemporal dementia with parkinsonism-17 (FTDP-17), argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, postencephalitic parkinsonism, prion protein cerebral amyloid angiopathy, progressive subcortical gliosis, progressive supranuclear palsy, subacute sclerosing panencephalitis, tangle only dementia, multi-infarct dementia and ischemic stroke; see for a review, e.g., Lee et al., Annu. Rev. Neurosci. 24 (2001), 1121-1159 in which Table 1 catalogs the unique members of tauopathies or Sergeant et al., Bioch. Biophy. Acta 1739 (2005), 179-97, with a list in FIG. 2 therein).

AD, the most common cause of dementia, is diagnosed by extracellular plaques containing β-amyloid (Aβ) peptides and intracellular neurofibrillary tangles (NFTs) from hyperphosphorylated, insoluble and filamentous tau proteins in neuropathology (Gao Y. L. et al., Ann Transl Med. 2018 May; 6(10): 175). In some embodiments, the disease is an amyloidosis-associated condition or Lewy Body Dementia. In other embodiments, the disease is Parkinson's Disease or Huntingtin disease (HD), which share as a hallmark, protein aggregates with fibrillary amyloid-like structures in the brain. These amyloid fibrils are composed of aggregation-prone proteins, such as mutant huntingtin (HTT) in Huntington disease, and α-synuclein in Parkinson disease (Stroo E et al., Front. Neurosci., 14 Feb. 2017, 11:64).

As used herein, the terms “treat”, “treating” and “treatment” and variations thereof refer to taking steps to obtain beneficial or desired results, including pharmacological and/or physiological results in a subject with neurodegenerative disease (e.g., Alzheimer's Disease (AD)). A treatment of this disclosure can reduce the severity of at least one discernible symptom of the neurodegenerative disease, or retard or slow the progression of at least one discernible symptom of neurodegenerative disease. A treatment of this disclosure can be prophylactic, i.e., it can partially or completely prevent the onset of the neurodegenerative disease (e.g., AD). Hence, the term “treatment” as used herein includes: (a) preventing the onset of AD in a subject who may be predisposed to AD, but has not yet been diagnosed as having it; (b) inhibiting AD and/or its symptoms; (c) ameliorating AD and/or its symptoms, e.g. causing regression of AD, or reducing cognitive or functional decline; (d) slowing the progression of AD and/or its symptoms; or (e) prolonging survival as compared to expected survival in an untreated control.

As used herein, the term “treats cognitive impairment”, “enhances cognitive function” and variations thereof refer to taking steps to improve cognitive function in a subject with cognitive impairment so that the subject's performance in one or more cognitive tests is improved to any detectable degree, or is prevented from further decline. Preferably, that subject's cognitive function, after treatment with an antibody of the disclosure, more closely resembles the function of a normal, unimpaired subject. In some cases, the subject's cognitive function is improved compared to the level of cognitive function in an untreated/placebo treated control subject and group of subjects. Treatment of cognitive impairment in humans may improve cognitive function to any detectable degree (e.g., at least about 20%, at least about 30%, at least about 40%, or at least about 50%, at least about 60%, at least about 70%, at least about 80%, or at least about 90%), but is preferably improved sufficiently to allow the impaired subject to carry out daily activities of normal life at the same level of proficiency as a normal, unimpaired subject. In some cases, “treating cognitive impairment” in a subject affecting by a neurodegenerative disorder (such as AD) refers to takings steps to improve cognitive function in the subject so that the subject's cognitive function, after treatment of cognitive impairment, more closely resembles the function of an age-matched normal, unimpaired subject, or the function of a young adult subject. In some cases, “treating cognitive impairment” in a subject refers to taking steps to delay or slow the progression of cognitive impairment in a subject with a neurodegenerative disease (such as AD). In some cases, “treating cognitive impairment” in a subject refers to taking steps to reduce the rate of decline of cognitive function in a subject with a neurodegenerative disease (such as AD).

In some embodiments, treatment results in symptomatic improvement. This includes but is not limited to enhanced cognition, more autonomy, and/or improvement in neuropsychiatric and behavioral dysfunction, any of which may be temporary or long-term.

As used herein, the term “control” refers to an age-matched subject that does not have or is not diagnosed with a neurodegenerative disorder. In some embodiments, a control refers to an age-matched and sex-matched subject that is not treated with the method of this disclosure, or is treated with a placebo. In some embodiments, a control refers to a population average for the amount or degree of a particular parameter in a normal healthy population.

As used herein, the terms “antibody” and “immunoglobulin” are used interchangeably. An antibody or immunoglobulin may be natural or partly or wholly synthetically produced, including but not limited to monoclonal antibodies, polyclonal antibodies, multispecific antibodies (for example, bispecific antibodies and polyreactive antibodies), and antibody fragments. Thus, the term “antibody” as used in any context within this specification is meant to include, but not be limited to, any specific binding member, immunoglobulin class and/or isotype (e.g., IgG1, IgG2a, IgG2b, IgG3, IgG4, IgM, IgA1, IgA2, IgD, and IgE) and biologically relevant fragment or specific binding member thereof, including but not limited to Fab, F(ab′)₂, scFv (single chain or related entity) and (scFv)₂.

As used herein, the term “antibody fragments” may include those antibody fragments obtained using techniques readily known and available to those of ordinary skill in the art, as reviewed herein. Therefore, in addition to the definition for “antibody” presented supra, the term “antibody” may further encompass any polypeptide or protein comprising a portion of an intact antibody, such as the antigen binding or variable region of the intact antibody. These can be derived from natural sources, or they may be partly or wholly synthetically produced. Examples of antibody fragments include, but are not limited to, Fab, Fab′, F(ab′)₂, and Fv fragments; diabodies, and linear antibodies.

As used herein, the term “epitope” may refer to the region of an antigen to which an antibody or T cell binds, e.g., a region within the beta (β) subunit of FSH, including but not limited to an epitope within SEQ ID NO: 1 or SEQ ID NO: 2. An “antigen” refers to a substance that elicits an immunological reaction or binds to the products of that reaction. The human FSHβ epitope is LVYKDPARPKIQK (SEQ ID NO: 1). The mouse FSHβ epitope is LVYKDPARPNTQK (SEQ ID NO: 2).

As used herein, the terms “follicle stimulating hormone” and/or “FSH” may refer to a gonadotropin, a type of glycoprotein polypeptide hormone. FSH is synthesized and secreted by the gonadotropic cells of the anterior pituitary gland and is implicated in regulating the development, growth, maturation, and reproductive processes of the body. FSH is a 35.5 kDa glycoprotein heterodimer, having two polypeptide units, an alpha (α) and beta (β) subunit. FSH is similar in structure to luteinizing hormone (LH), thyroid stimulating hormone (TSH), and human chorionic gonadotropin (hCG), sharing an identical alpha (α) subunit, but having variations in the beta (β) subunit. This makes the beta (β) subunit an attractive therapeutic target for FSH inhibitors as the inhibitors targeting the beta (β) subunit, e.g., one or more epitopes located within the beta (β) subunit, can be specific to inhibiting FSH. An exemplary gene encoding the beta (β) subunit of human FSH may be accessed at, e.g., Accession No. NM _ 000510. An exemplary gene encoding the beta (β) subunit of murine FSH may be accessed at, e.g., NM 008045. One of ordinary skill in the art will be able to reach predicted amino acid sequences from the provided nucleotide sequences.

Follicle Stimulating Hormone is known to rise sharply to counter the declining ovarian reserve (Randolph, J. F., Jr. et al. J Clin Endocrinol Metab 88, 1516-1522, (2003)). before the last menstrual period in a human female when estrogen levels are relatively unperturbed. During this perimenopausal phase, normally between age 45 and 54 years (Ref 8), certain neuropathologic and cognitive features display a ‘spike’, particularly when compared to pre-or post-menopausal women (Dubois, B. et al. Alzheimers Dement 12, 292-323, (2016); Epperson, C. N., et al. J Clin Endocrinol Metab 98, 3829-3838, (2013); Hampel, H. et al. Front Neuroendocrinol 50, 31-51, (2018); Jack, C. R., Jr. et al. Lancet Neurol 12, 207-216, (2013)). In addition, high serum FSH levels (>60 IU/L) in post-menopausal women are strongly associated with the onset of AD (Short, R. A., et al. Elevated gonadotropin levels in patients with Alzheimer disease. Mayo Clin Proc 76, 906-909, (2001); Bowen, R. L., et al. J Neuroendocrinol 12, 351-354, (2000)).

As used herein, the terms “follicle stimulating hormone receptor” and/or “FSHR” may refer to a transmembrane receptor that interacts with FSH. FSHR is a G protein coupled receptor (GPCR). Activation of FSHR is necessary for the hormonal functioning of FSH. The B subunit of FSH is necessary for binding to FSHR, and thus the β subunit confers upon FSH its specific biological action. Therefore, because the biological activity of FSH relies upon binding to FSHR, inhibiting the biological activity of FSH may be achieved either by directly inactivating FSH, e.g., by binding to the β subunit of FSH, or by directly inactivating FSHR. This is because inactivating FSHR will result in a loss of biological activity similar to that of inactivating FSH as the biological activity relies on binding between FSH and FSHR. An exemplary gene encoding human FSHR may be accessed at, e.g., Accession No. XM 011532734 (transcript variant X2) or XM_011532733 (transcript variant X1). An exemplary gene encoding FSHR may be accessed at, e.g., Accession No. NM_013523.3. One of ordinary skill in the art will be able to reach predicted amino acid sequences from the provided nucleotide sequences.

As used herein, the term “subject” or “individual” or “animal” or “patient” is meant any subject, particularly a mammalian subject, e.g., a human patient, for whom diagnosis, prognosis, prevention, or therapy is desired. In some embodiments, the term “patient” means a female patient. In some embodiments, the term “patient” means a male patient. In some embodiments, the term “patient” means a menopausal female patient. In some embodiments, the term “patient” means a premenopausal female patient. In some embodiments, the term “patient” means a perimenopausal female patient.

As used herein, the term “administering” or variants thereof, refer to dispensing or delivering the composition of this disclosure by using one of a variety of methods known to those skilled in the art. For example, an antibody or composition comprising an antibody of this disclosure can be administered subcutaneously, intramuscularly, intravenously, intrathecally, or intracranially to the subject. Administering can also be performed, for example, once, a plurality of times, and/or over one or more extended periods. In some aspects, the administration includes both direct administration, including self-administration, and indirect administration, including the act of prescribing a drug or composition. For example, as used herein, a physician who instructs a patient to self-administer a composition, or to have the composition administered by another and/or who provides a patient with a prescription for administering the composition to the patient.

Appropriate methods of administering a composition or antibody of the disclosure to a subject will also depend, for example, on the age of the subject, whether the subject is active or inactive at the time of administering, whether the subject is showing symptoms of the neurodegenerative disorder, the extent of cognitive impairment, and the chemical and biological properties of the composition (e.g., solubility, digestibility, bioavailability, stability and toxicity). In some embodiments, a composition of the disclosure is administered intravenously, e.g., to a subject by injection. The antibody of the disclosure is typically administered to the patient by intravenous infusion following dilution into saline.

As used herein, the term “carriers” may include pharmaceutically acceptable carriers, excipients, or stabilizers that are nontoxic to the cell or mammal being exposed thereto at the dosages and concentrations employed. Often the pharmaceutically acceptable carrier is an aqueous pH buffered solution. Examples of physiologically acceptable carriers include, but not limited to, buffers such as phosphate, citrate, and other organic acids; antioxidants including, but not limited to, ascorbic acid; low molecular weight (less than about 10 residues) polypeptide; proteins, such as, but not limited to, serum albumin, gelatin, or immunoglobulins; hydrophilic polymers such as, but not limited to, polyvinylpyrrolidone; amino acids such as, but not limited to, glycine, glutamine, asparagine, arginine or lysine; monosaccharides, disaccharides, and other carbohydrates including, but not limited to, glucose, mannose, or dextrins; chelating agents such as, but not limited to, EDTA; sugar alcohols such as, but not limited to, mannitol or sorbitol; salt-forming counterions such as, but not limited to, sodium; and/or nonionic surfactants such as, but not limited to, TWEEN; polyethylene glycol (PEG), and PLURONICS. Any combination of such components, including probable inclusion of a bacteriostat, may be useful to fill the formulations of the present disclosure.

As used herein, the term “homology” may refer to the existence of shared structure between two compositions. The term “homology” in the context of proteins may refer to the amount (e.g., expressed in a percentage) of overlap between two or more amino acid and/or peptide sequences. The “percent identity” between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm. Such homology is well-represented in the art via local alignment tools and/or algorithms, and may include pairwise alignment, multiple sequence alignment methods, structural alignment methods, and/or phylogenetic analysis methods. Where sequences differ in conservative substitutions, the percent sequence identity may be, but not necessarily is, adjusted upwards to correct for the conservative nature of the substitution. Means for making this adjustment are well known to those of skill in the art. Typically, but not necessarily, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1.

Generally, variants of a particular polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polypeptide as determined by sequence alignment programs and parameters described herein and known to those skilled in the art. Tools for sequence alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). For instance, the percent identity between two amino acid sequences may be determined using the Needleman-Wunsch algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. Those skilled in the art can determine appropriate parameters for measuring alignment, including any algorithms needed to achieve maximal alignment over the full length of the sequences being compared. Sequence identity is then calculated relative to the longer sequence, i.e., even if a shorter sequence shows 100% sequence identity with a portion of a longer sequence, the overall sequence identity will be less than 100%. Unless otherwise stated the BLASTP program (for amino acid sequences), which uses as defaults a word length (W) of 3, and expectation (E) of 10, and the BLOSUM62 scoring matrix (see Henikoff & Henikoff, Proc. Natl. Acad. Sci. USA 89:10915, 1989) should be used to determine percent identity.

The terms “conservative sequence modifications” or “conservative substitutions” as used herein may refer to amino acid modifications to a target epitope or antibodies and antigen-binding portions thereof of the disclosure that does not significantly affect or alter the binding characteristics of the anti-FSH antibodies, for example but not necessarily Hf2, Hu6, Hu26, or Hu28, and antigen-binding portions thereof, including but not limited to SEQ ID NO: 1 and SEQ ID NO: 2. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a target epitope that the anti-FSH antibodies of the disclosure specifically bind to, e.g., epitopes on the beta (β) subunit of FSH for some anti-FSH antibodies, can be replaced with other amino acid residues from the same side chain family and the antibodies of the present disclosure can be tested against the target epitope can be tested, for example using functional assays described herein or otherwise known in the art. Likewise, one or more amino acid residues within the CDR regions of an antibody of the disclosure, e.g., Hf2, Hu6, Hu26, or Hu28, can be replaced with other amino acid residues from the same side chain family and the altered antibody can be tested for retained function using the functional assays described herein.

### Anti-FSH Antibodies and Antigen-Binding Fragments Thereof

The antibodies of the present disclosure are anti-FSH antibodies or antigen-binding fragments thereof. An anti-FSH antibody may take one of numerous forms in the art, as disclosed herein. Antibodies are in part defined by the antigens to which they bind, thus, an “anti-FSH antibody” is any such antibody which specifically binds at least one epitope found on FSH. In some embodiments, the epitope is located in the β subunit of FSH. In some embodiments, the epitope is located within LVYKDPARPKIQK (SEQ ID NO: 1). In some embodiments, the epitope is located within LVYKDPARPNTQK (SEQ ID NO: 2). It is understood in the art that an antibody is a glycoprotein comprising at least two heavy (H) chains and two light (L) chains inter-connected by disulfide bonds, or an antigen binding portion thereof. A heavy chain comprises a heavy chain variable region (VH) and a heavy chain constant region (CH1, CH2 and CH3). A light chain comprises a light chain variable region (VL) and a light chain constant region (CL). The variable regions of both the heavy and light chains comprise framework regions (FWR) and complementarity determining regions (CDR). The four FWR regions are relatively conserved while CDR regions (CDR1, CDR2 and CDR3) represent hypervariable regions and are arranged from NH, terminus to the COOH terminus as follows: FWR1, CDR1, FWR2, CDR2, FWR3, CDR3, FWR4. The variable regions of the heavy and light chains contain a binding domain that interacts with an antigen while, depending of the isotype, the constant region(s) may mediate the binding of the immunoglobulin to host tissues or factors. It is known in the art that it is possible to manipulate monoclonal and other antibodies and use techniques of recombinant DNA technology to produce other antibodies or chimeric molecules which retain the specificity of the original antibody. Such techniques may evolve introducing DNA encoding the immunoglobulin variable region, or CDRs, of an antibody to the constant regions, or constant regions plus framework regions, of a different immunoglobulin.

CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis (www.bioinf.org.uk/abysis/sequence_input/key_annotation/key_annotation.cgi)) are available and known to those of skill in the art for analysis of antibody sequences and determination of CDRs. The specific CDR sequences defined herein are generally based on Kabat definitions. However, it will be understood that reference to a heavy chain CDR or CDRs and/or a light chain CDR or CDRs of a specific antibody will encompass all CDR definitions as known to those of skill in the art.

An exemplary anti-FSH antibody or antigen-binding portion thereof of the present disclosure comprises Hf2 or antigen binding portion thereof exemplified in US20190241651, incorporated by reference herein in its entirety. Hf2 has a variable heavy chain region comprising SEQ ID NO: 3 and a variable light chain region comprising SEQ ID NO: 4. Hf2 has a CDRH1 comprising SEQ ID NO: 5, a CDRH2 comprising SEQ ID NO: 6, a CDRH3 comprising SEQ ID NO: 7, a CDRL1 comprising SEQ ID NO: 8, a CDRL2 comprising SEQ ID NO: 9, and a CDRL3 comprising SEQ ID NO: 10. However, the anti-FSH antibodies or antigen-binding portions thereof of the present disclosure are not limited as such. For example, in some embodiments, the anti-FSH antibody has a variable heavy chain region having at least 70%, at least 75%, at least 80%, at least 85%, at least 90%, at least 95%, or at least 99% identity with SEQ ID NO: 3. In some embodiments, SEQ ID NO: 3 has at least one conservative substitution. In some embodiments, the anti-FSH antibody has a variable light chain region having at least 70%, at least 75%, at least 80%, at least 85%, at least 90%, at least 95%, or at least 99% identity with SEQ ID NO: 4. In some embodiments, SEQ ID NO: 4 has at least one conservative substitution. In some embodiments, one or more of the CDRs of the variable heavy chain region has at least 70%, at least 75%, at least 80%, at least 85%, at least 90%, at least 95%, or at least 99% identity with one or more of SEQ ID NO: 5, SEQ ID NO: 6, and SEQ ID NO: 7. In some embodiments, one or more of SEO ID NO: 5, SEQ ID NO: 6, and SEQ ID NO: 7 has at least one conservative substitution. In some embodiments, one or more of the CDRs of the variable light chain region has at least 70%, at least 75%, at least 80%, at least 85%, at least 90%, at least 95%, or at least 99% identity with one or more of SEQ ID NO: 8, SEQ ID NO: 9, and SEQ ID NO: 10. In some embodiments, one or more of SEQ ID NO: 8, SEQ ID NO: 9, and SEQ ID NO: 10 has at least one conservative substitution.

Another exemplary anti-FSH antibody or antigen-binding portion thereof of the present disclosure comprises Hu6 or antigen binding portion thereof exemplified in Gera S. et al., PNAS, 117(46):28971-79. Hu6 has a variable heavy chain region comprising SEQ ID NO: 11 and a variable light chain region comprising SEQ ID NO: 12. Hu6 has a CDRH1 comprising SEQ ID NO: 5, a CDRH2 comprising SEQ ID NO: 6, a CDRH3 comprising SEQ ID NO: 7, a CDRL1 comprising SEQ ID NO: 8, a CDRL2 comprising SEQ ID NO: 9, and a CDRL3 comprising SEQ ID NO: 10. In some embodiments, the anti-FSH antibody has a variable heavy chain region having at least 70%, at least 75%, at least 80%, at least 85%, at least 90%, at least 95%, or at least 99% identity with SEQ ID NO: 11. In some embodiments, SEQ ID NO: 11 has at least one conservative substitution. In some embodiments, the anti-FSH antibody has a variable light chain region having at least 70%, at least 75%, at least 80%, at least 85%, at least 90%, at least 95%, or at least 99% identity with SEQ ID NO: 12. In some embodiments, SEQ ID NO: 12 has at least one conservative substitution.

Another exemplary anti-FSH antibody or antigen-binding portion thereof of the present disclosure comprises Hu26 or antigen binding portion thereof exemplified in Gera S. et al., PNAS, 117(46):28971-79. Hu26 has a variable heavy chain region comprising SEQ ID NO: 13 and a variable light chain region comprising SEQ ID NO: 14. Hu26 has a CDRH1 comprising SEQ ID NO: 5, a CDRH2 comprising SEQ ID NO: 6, a CDRH3 comprising SEQ ID NO: 7, a CDRL1 comprising SEQ ID NO: 8, a CDRL2 comprising SEQ ID NO: 9, and a CDRL3 comprising SEQ ID NO: 10. Yet another exemplary anti-FSH antibody or antigen-binding portion thereof of the present disclosure comprises Hu28 or antigen binding portion thereof exemplified in Gera S. et al., PNAS, 117(46):28971-79. Hu28 has a variable heavy chain region comprising SEQ ID NO: 15 and a variable light chain region comprising SEQ ID NO: 16. Hu28 has a CDRH1 comprising SEQ ID NO: 5, a CDRH2 comprising SEQ ID NO: 6, a CDRH3 comprising SEQ ID NO: 7, a CDRL1 comprising SEQ ID NO: 8, a CDRL2 comprising SEQ ID NO: 9, and a CDRL3 comprising SEQ ID NO: 10.

The CDRs for the exemplified anti-FSH antibodies of this disclosure are as follows in Table 1:

TABLE 1
CDR sequences

| Antibody | | |
|---|---|---|
| clone | Type of CDR | CDRs (Kabat) |
| Hf2/Hu6/ | Heavy chain CDR1 | SDYAWN (SEQ ID NO: 5) |
| Hu26/Hu28 | Heavy chain CDR2 | SIFSSGSINYNPSLKS |
| | | (SEQ ID NO: 6) |
| | Heavy chain CDR3 | GGTGTDY |
| | | (SEQ ID NO: 7) |
| Hf2/Hu6/ | Light chain CDR1 | RASQDISNYLS |
| Hu26/Hu28 | | (SEQ ID NO: 8) |
| | Light chain CDR2 | YTSRLHS |
| | | (SEQ ID NO: 9) |
| | Light chain CDR3 | QQGHTLPPT |
| | | (SEQ ID NO: 10) |

The Variable Heavy (VH) and Variable Light (VL) chain sequences for the exemplified anti-FSH antibodies of this disclosure are as follows in Table 2:

TABLE 2
VH and VL sequences of the anti-FSH
antibodies of this disclosure

| Antibody clone | VH/VL sequence |
|---|---|
| Hf2 VH | DVHLQESGPGLVKPSQSLSLTCTVTGFSITSDYAWNWIR |
| | QFPGNKLEWMGSIFSSGSINYNPSLKSRISITRDTSRNQF |
| | FLQLNSVTTADAGTYYCARGGTGTDYWGQGTTLTVSS |
| | (SEQ ID NO: 3) |
| Hf2 VL | DIQMTQTTSSLSASLGDRVTISCRASQDISNYLSWYQQK |
| | PDGTIKLLIYYTSRLHSGVSSRFSGSGSGTDYSLTISNLE |
| | QEDFATYFCQQGHTLPPTFGGGTKLEIK |
| | (SEQ ID NO: 4) |
| Hu6 VH | QVQLQESGPGLVKPSQTLSLTCTVSGFSITSDYAWNWIR |
| | QPPGKGLEWIGSIFSSGSINYNPSLKSRVTISRDTSKNQF |
| | SLKLSSVTAADTAVYYCARGGTGTDYWGQGTLVTVSS |
| | (SEQ ID NO: 11) |
| Hu6 VL | DIQMTQSPSSLSASVGDRVTITCRASQDISNYLSWYQQK |
| | PGKAIKLLIYYTSRLHSGVPSRFSGSGSGTDYTLTISSLQ |
| | PEDFATYFCQQGHTLPPTFGGGTKVEIK |
| | (SEQ ID NO: 12) |
| Hu26 VH | QVQLQESGPGLVKPSQTLSLTCTVSGFSITSDYAWNWIR |
| | QPPGKGLEWIGSIFSSGSINYNPSLKSRVTISRDTSKNQF |
| | SLKLSSVTAADTGVYYCARGGTGTDYWGQGTLVTVSS |
| | (SEQ ID NO: 13) |
| Hu26 VL | DIQMTQSPSSLSASVGDRVTITCRASQDISNYLSWYQQK |
| | PGGAPKLLIYYTSRLHSGVPSRFSGSGSGTDYTLTISSLQ |
| | PEDFATYFCQQGHTLPPTFGGGTKVEIK |
| | (SEQ ID NO: 14) |
| Hu28 VH | QVQLQESGPGLVKPSQTLSLTCTVSGFSITSDYAWNWIR |
| | QPPGKGLEWMGSIFSSGSINYNPSLKSRITISRDTSKNQF |
| | SLKLSSVTAADTGVYYCARGGTGTDYWGQGTLVTVSS |
| | (SEQ ID NO: 15) |
| Hu28 VL | DIQMTQSPSSLSASVGDRVTITCRASQDISNYLSWYQQK |
| | PGGAPKLLIYYTSRLHSGVPSRFSGSGSGTDYTLTISSLQ |
| | PEDFATYFCQQGHTLPPTFGGGTKVEIK |
| | (SEQ ID NO: 16) |

In some embodiments, the anti-FSH antibodies comprise the following CDR sequences shown in Table 3:

TABLE 3
CDR sequences

| Type of CDR | CDRs (Kabat) |
|---|---|
| Heavy chain CDR1 | SDYAWN |
| | (SEQ ID NO: 5) |
| Heavy chain CDR2 | SIFSSGSINYNPSLKS |
| | (SEQ ID NO: 6) |
| Heavy chain CDR3 | GGTGTDY |
| | (SEQ ID NO: 7) |
| Light chain CDR1 | RASQDISNYLS |
| | (SEQ ID NO: 8) |
| Light chain CDR2 | YTSRLHS |
| | (SEQ ID NO: 9) |
| Light chain CDR3 | QQGHTLPPT |
| | (SEQ ID NO: 10) |

The antibodies of the disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the FSH polypeptide or a variant thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the FSH polypeptide or a variant thereof. The immunizing agent may comprise an antigenic fragment of the beta (β) subunit of FSH. The immunizing agent may be a peptide sequence comprising SEQ ID NO: 1 or SEQ ID NO: 2 or a peptide sequence consisting essentially of SEQ ID NO: 1 or SEQ ID NO:2 but having conservative substitutions. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63). The culture medium in which the hybridoma cells are cultured can then be assayed for the presence of monoclonal antibodies directed against FSH. Preferably, the binding specificity of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the disclosure can be readily isolated and sequenced using conventional procedures (e.g., by using oligonucleotide probes that are capable of binding specifically to genes encoding the heavy and light chains of murine antibodies). The hybridoma cells of the disclosure serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, as in U.S. Pat. No. 4,816,567, or by covalently joining to the immunoglobulin coding sequence all or part of the coding sequence for a non-immunoglobulin polypeptide. Such a non-immunoglobulin polypeptide can be substituted for the constant domains of an antibody of the disclosure, or can be substituted for the variable domains of one antigen-combining site of an antibody of the disclosure to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

In some embodiments, the antibodies or antigen binding fragments thereof of the present disclosure are covalently ligated to or enclosed within liposomes, albumin microspheres, microemulsions, nano-particles or nanocapsules. In some embodiments, the antibodies or antigen binding fragments thereof of the present disclosure are covalently ligated to a blood brain barrier protein, or conjugated to an antibody which binds to a receptor on blood vessels (such as an antibody to transferrin receptor (TfR), insulin receptor, leptin receptor, lipoprotein receptor, IGF receptor, LDL Receptor, P-selectin (CD62P), intracellular adhesion molecule-1 (ICAM-1, or CD54), or other targets for cerebrovascular targeting), in order to facilitate blood-brain barrier penetration and delivery of the anti-FSH antibody or antigen binding fragment thereof to the disease site. Blood brain barrier shuttles, such as those disclosed in WO2014033074 may be used to deliver the anti-FSH antibody or antigen binding fragment thereof to the brain.

### Pharmaceutical Compositions

The anti-FSH antibody of the present disclosure can be formulated as a pharmaceutical composition. Such pharmaceutical compositions can be formulated according to methods well known in the art; see, for example, Remington: The Science and Practice of Pharmacy (2000) by the University of Sciences in Philadelphia, ISBN 683-306472. The compositions can further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Additionally, the pharmaceutical compositions for injection may be prepared in lipophilic solvents, which include, but is not limited to, oils, such as vegetable oils, olive oil, peanut oil, palm oil soybean oil, safflower oil, etc.; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions, (see for example, Liu, F. et al. Pharm. Res. 12:1060-1064 (1995); Prankerd, R. J. J. Parent. Sci. Tech. 44:139-49 (1990); U.S. Pat. No. 5,651,991).

In some embodiments, the anti-FSH antibody of the present disclosure can be formulated as a pharmaceutical composition including, for example, one or more buffers, one or more salts, one or more surfactants, one or more sugars, and/or one or more lyoprotectants/cryoprotectants. The anti-FSH antibody formulation can include phosphate at a molar concentration of about 5 mM, about 10 mM, about 15 mM, about 15 mM, about 20 mM, about 25 mM, about 30 mM, about 35 mM, about 40 mM, about 45 mM, or about 50 mM. The anti-FSH antibody formulation can include Tween-20 at a concentration of about 0.001%, about 0.002%, about 0.003%, about 0.004%, or about 0.005% (v/v). The anti-FSH antibody formulation can include sodium chloride (NaCl) at a molar concentration of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 2.0 mM, about 3.0 mM, about 4.0 mM, or about 5.0 mM. The anti-FSH antibody formulation can include sucrose at a molar concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, or about 300 mM.

The pharmaceutical composition may comprise additional agents. For example, for use in the treatment of Alzheimer's disease the additional agent can be selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of NGF, CERE-110, beta-blocker, human amyloid vaccine, beta or gamma secretase inhibitor, nicotinic or muscarinic agonist, and a second antibody, such as an anti-Aβ antibodies, anti-Tau antibodies, and combinations thereof.

The compositions of the disclosure can be administered through various routes known in the art, e.g., by intravenous, intraperitoneal, subcutaneous, intramuscular, topical, or intradermal administration.

The dose and dosage regimen depends upon a variety of factors readily determined by a physician, such as the severity of the condition, the patient, and the patient's history. Generally, a therapeutically effective amount of a composition is administered to a patient. In some embodiments, the amount of composition administered is in the range of about 0.1 mg/kg to about 100 mg/kg of patient body weight, and any range in between. Depending on the severity of condition, about 0.1 mg/kg to about 50 mg/kg body weight (for example, about 0.1-15 mg/kg/dose, more usually from about 0.2-25 mg/kg body weight) of composition is an initial candidate dosage for administration to the patient, whether, for example, by one or more separate administrations, or by continuous infusion. The compositions may be delivered relatively low volume rates, for example but not necessarily from about 0.001 ml/day to 10 ml/day so as to minimize tissue disturbance or trauma near the site where the formulation is released. The formulation may be released at a rate of, depending on the specific biological agent(s), at a low dose, e.g., from about 0.01 μg/hr or 0.1 μg/hr, 0.25 μg/hr, 1 μg/hr, generally up to about 200 μg/hr, or the formulation is delivered at a low volume rate e.g., a volume rate of from about 0.001 ml/day to about 1 ml/day, for example, 0.01 micrograms per day up to about 20 milligrams per day. Dosage depends on a number of factors such as potency, bioavailability, and toxicity of the active ingredient and the requirements of the subject. The progress of this therapy is readily monitored by conventional methods and assays and based on criteria known to the physician or other persons of skill in the art. The above parameters for assessing successful treatment and improvement in the disease are readily measurable by routine procedures familiar to a physician.

### Methods of Treatment

The methods disclosed herein enable the treatment of neurodegenerative disorders (such as Alzheimer's Disease) in subjects with compositions of the disclosure. A composition of the disclosure comprises a recombinant, fully human, anti-FSH monoclonal antibody administered to the human patient. In a preferred embodiment, the monoclonal antibody has an excellent safety profile while being selective for FSH. A preferred monoclonal antibody meeting these criteria is the antibody comprising CDRs of SEQ ID NOs: 5-10. The antibody of the disclosure is a biologic treatment for Alzheimer's disease which is a non-naturally occurring, recombinant, fully human, anti-FSH monoclonal antibody that recognizes FSH, including plaques. A preferred antibody of the disclosure is an IgG1 consisting of 2 heavy and 2 kappa light chains connected by inter-chain disulfide.

The methods of this disclosure further comprise administration of an anti-FSH antibody in combination with an additional therapeutic agent for a neurodegenerative disease (e.g., AD). In some embodiments, an antibody of the disclosure is concurrently administered with one or more agents including but not limited to cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of NGF, CERE-110, beta-blocker, human amyloid vaccine, beta or gamma secretase inhibitor, nicotinic or muscarinic agonist, and a second antibody. The cholinesterase inhibitor includes but is not limited to galantamine, rivastigmine, tacrine, and donepezil. The NMDA receptor antagonist includes but is not limited to ketamine, methadone, memantine, amantadine, and dextromethorphan or a salt thereof. The antipsychotic agent includes but is not limited to aripiprazole, risperidone, olanzapine, quetiapine, or haloperidol. The benzodiazepine includes but is not limited to lorazepam, oxazepam and temazepam. The tricyclic antidepressant includes but is not limited to nortriptyline. The hormone includes but is not limited to estrogen, progesterone and leuprolide. The vitamin includes but is not limited to folate and nicotinamide. The second antibody includes but is not limited to bapineuzumab, solanezumab, gantenerumab, crenezumab, ponezumab, BAN2401, and aducanumab.

Furthermore, any therapy described herein can include one or more agents for treating, one or more side-effects of a therapy comprising the neurodegenerative disease therapy. Combination therapies (e.g., co-administration of an anti-FSH antibody composition and one or more additional neurodegenerative therapies or additional therapeutic agents) can be, e.g., simultaneous or successive. For example, the anti-FSH antibody of this disclosure and the additional therapeutic agent(s) can be administered at the same time or at different times. In some embodiments, the one or more additional therapeutic agents can be administered first in time and the anti-FSH antibody can be administered second in time.

### Animal Models

Animal models serve as an important resource for developing and evaluating treatments for neurodegenerative disorders. Features that characterize pathological hallmarks (such as amyloid deposits and neurofibrillary tangles) and cognitive impairment in animal models typically extend to pathological hallmarks cognitive impairment in humans (LaFerla F M, Green K N. Cold Spring Harb Perspect Med. 2012; 2(11):a006320. Published 2012 Nov. 1.). Efficacy in such animal models is, thus, expected to be predictive of efficacy in humans.

In some embodiments, an animal model of neurodegenerative disease can express one or more human genes. Animal models of Alzheimer's disease can include one or more mutations in one or more genes, for example, in amyloid precursor protein (APP, e.g., human transgene APP models with the Swedish mutation (K670D/M671L), the Indiana mutation (V717F), the London mutation (V7171), and/or the Arctic mutation (E693G)), presenilin 1 (PSEN1), presnilin 2 (PSEN2), tau (MAPT, e.g. N279K, AK280, P301L, P301S, V337 and/or R406W), apolipoprotein E (APOE), progranulin (PGRN), TAR DNA-binding protein TDP-43 (TARDBP), valosin-containing protein (VCP). Non-limiting examples of animal models of Alzheimer's disease include mouse models (e.g., PDAPP, H6, J9, J20, Tg2576, APP23, C3-3, CRND8, ARC6/ARC48, C3-3 x PSEN1, PSAPP, APP^{SL}PS1^{M146L}, 5XFAD, hBACE1/hAPP, hTau, Tau^{P301L}, Tau^{V337M}, Tau^{P301S}, Tau^{G272V, P301S}, 3xTg-AD). In some embodiments, an animal model can be the progeny of a wild-type (wt) animal and a transgenic (tg) animal. Various animal models of neurodegenerative diseases are known in the art, such as the PD APP, Tg2576, APP23, TgCRNDS, J20, hPS2 Tg, and APP +PS1 transgenic mice. Sankaranarayanan, Curr. Top. Medicinal Chem. 6:609-627, 2006; Kobayashi et al. Genes Brain Behav. 4:173-196. 2005; Ashe and Zahns, Neuron. 66:631-45, 2010. Such animal models of dementia may be used to assay the effectiveness of the methods and compositions of this disclosure in treating neurodegenerative disease.

In some embodiments, animal models may be used to evaluate the safety of a pharmaceutical composition, for example, to evaluate the safety of a pharmaceutical composition of an antibody therapy or combination therapy comprising antibodies and one or more additional therapeutic agents. The use of animal models for evaluating the safety of a pharmaceutical composition can include investigating pharmacokinetics, pharmacodynamics, toxicology, efficacy, embryonic toxicity, carcinogenic potential, among other measures of safety of a pharmaceutical composition. Animal models for evaluating the safety of a pharmaceutical composition may include, for example, the use of mice, fish, frogs, rabbits, cats, dogs, or non-human primates.

### Cognitive Tests in Animal Models and Humans

The extent of cognitive impairment in an animal model for a neurodegenerative disorder, and the efficacy of a method of treatment for said neurodegenerative disorder may be tested and confirmed with the use of a variety of cognitive tests.

Novel Object Recognition (NOR) Test: In this test for recognition memory, a mouse is presented with two identical objects during the first session, and then one of the two objects is replaced by a novel object during a second session (Leger, M. et al. Nat Protoc 8, 2531-2537, (2013)). In one example, the test is conducted for induced preference and discriminating ability; the latter should be >0.25. On day 1, a habituation phase in an empty arena (for 5 to 10 minutes) is followed 24 hours later by the training phase, which allows for a 5- to-10-min exploration in the habituated arena where two identical objects are placed in opposite quadrants. The testing phase, which follows a 20-min to 4-hr retention time, is followed by replacement of one object with a novel object and 5 to 10 min of exploration. Parameters, including time spent, distance and number of head entries into quadrants, and object sniffing time, are collected using an ANY-Maze Video Tracking System (Stoelting, UK).

Fear Conditioning Test: The Fear Conditioning Test measures acquisition (Kim, J. J. & Fanselow, M. S. Science 256, 675-677, (1992); Maren, S., et al., Behav Brain Res 88, 261-274, (1997)) and retrieval (Anagnostaras, S. G., et al., J Neurosci 19, 1106-1114 (1999)) of both remote and recent context-related fear. In one example, for remote memory, mice, when young, will receive context conditioning consisting of one foot shock (1.0 mA constant current, 2 sec) per day for three days. They then undergo remote memory recall (20 min), with freezing behavior measured every 4 months until death or sacrifice (as in Amadi, U. et al., Psychol Sci 28, 143-161, (2017); Harmatz, E. S. et al. Biol Psychiatry 81, (2017)). For recent contextual memory retrieval, context conditioning is administered in a new context, with memory recall 24-h post-conditioning.

Radial Arm Maze Test: A Radial Arm Maze (RAM) behavioral task is another example of a cognitive test, specifically testing spatial memory (Chappeli et al. Neuropharmacology 37: 481-487, 1998). The RAM apparatus consists of, e.g., eight equidistantly spaced arms. A maze arm projects from each facet of a center platform. A food well is located at the distal end of each arm. Food is used as a reward. Blocks can be positioned to prevent entry to any arm. Numerous extra maze cues surrounding the apparatus may also be provided. After habituation and training phases, spatial memory of the subjects may be tested in the RAM under control or test compound-treated conditions. As a part of the test, subjects are pretreated before trial s with a vehicle control or one of a range of dosages of the test compound. At the beginning of each trial, a subset of the arms of the eight-arm maze is blocked. Subjects are allowed to obtain food on the unblocked arms to which access is permitted during this initial “information phase” of the trial. Subjects are then removed from the maze for a delay period, e.g., a 60 second delay, a 15 minute delay, a one-hour delay, a two-hour delay, a six hour delay, a 24 hour delay, or longer) between the information phase and the subsequent “retention test,” during which the barriers on the maze are removed, thus allowing access to all eight arms. After the delay period, subjects are placed back onto the center platform (with the barriers to the previously blocked arms removed) and allowed to obtain the remaining food rewards during this retention test phase of the trial. The identity and configuration of the blocked arms vary across trials. The number of “errors” the subjects make during the retention test phase is tracked. An error occurs in the trial if the subjects entered an arm from which food had already been retrieved in the pre-delay component of the trial, or if it re-visits an arm in the post-delay session that had already been visited. A fewer number of errors would indicate better spatial memory. The number of errors made by the test subject, under various test compound treatment regimes, can then be compared for efficacy of the test compound (e.g., the anti-FSH antibody of the present disclosure) in treating neurodegenerative disorders with cognitive impairment.

Morris Water Maze Test: Another cognitive test that may be used to assess the effects of a test compound on the cognitive impairment of a neurodegenerative disorder model animal is the Morris water maze. A water maze is a pool surrounded with a novel set of patterns relative to the maze. The training protocol for the water maze may be based on a modified water maze task that has been shown to be hippocampal-dependent (de Hoz et al, Eur. J. Neurosci., 22:745-54, 2005; Steele and Morris, Hippocampus 9: 118-36, 1999). The subject is trained to locate a submerged escape platform hidden underneath the surface of the pool. During the training trial, a subject is released in the maze (pool) from random starting positions around the perimeter of the pool. The starting position varies from trial to trial. If the subject does not locate the escape platform within a set time, the experimenter guides and places the subject on the platform to “teach” the location of the platform. After a delay period following the last training trial, a retention test in the absence of the escape platform is given to assess spatial memory. The subject's level of preference for the location of the (now absent) escape platform, as measured by, e.g., the time spent in that location or the number of crossings of that location made by the mouse, indicates better spatial memory, i.e., treatment of cognitive impairment. The preference for the location of the escape platform under different treatment conditions, can then be compared for efficacy of the test compound (e.g., the anti-FSH antibody of the present disclosure) in treating neurodegenerative disorders with cognitive impairment.

Other Tests in Animal Models: Other tests for evaluating cognitive function in animal models include the Five-choice Serial Reaction Time Test, (The 5-choice serial reaction time task: behavioural pharmacology and functional neurochemistry. Psychopharmacology (Berl). 2002 October; 163(3-4): 362-80). Fear Conditioning Paradigm (Gould T J et al., 2002 Behav Pharmacol. 13(4):287-94; Hamm A O et al., 2003 Brain 126 (Pt 2):267-75) and novel object recognition (Animal Models of Cognitive Impairment, 2006 (Ed. By Levin, E. D. & Buccafusco, J. J.). Boca Raton, Fla.).

Tests in Humans: There are various tests known in the art for assessing cognitive function in humans, for example and without limitation, the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest: and the explicit 3-alternative forced choice task (see, e.g., WO2012109491); the Visual Reproduction subtest of the Wechsler Memory Scale-Revised (WMS-R) (Wechsler, 1997); the Benton Visual Retention Test. See Folstein et al., J Psychiatric Res 12: 189-98, (1975); Robbins et al., Dementia 5: 266-81, (1994); Rey, L'examen clinique en psychologie, (1964); luger et al., J Geriair Psychiatry Neurol 12:168-79, (1999); Marquis et al, 2002 and Masur et al,, 1994,

### Assessment Methods for Alzheimer's Disease

Measurement of the risk, existence, severity, and progression of a neurodegenerative disease of this disclosure (e.g., AD) can be determined by clinical diagnosis over time; assessment of the global functional level of the patient; evaluation of the daily living capacities or behavioral deficits; volumetric analysis of brain structures; in vivo measurement of pathological deposits of abnormal proteins in brain (e.g. PET beta-amyloid imaging), or biochemical variables in body fluids (e.g. tau proteins or Abeta peptides); and by comparison to the natural course/history of the disease. In some embodiments, the following clinical assessments can be employed in determining the stage of Alzheimer's disease in the patient: Clinical Dementia Rating (CDR), the Free and Cued Selective Reminding Test (FCSRT), Neuropsychiatry Inventory-Questionnaire (NPI-Q), and a neuropsychological test battery comprising Rey Auditory Verbal Learning Test (RA VLT) Immediate and Delayed Recall, Wechsler Memory Scale (WMS) Verbal Pair Associate Learning Test Immediate and Delayed Recall, Delis-Kaplan Executive Function System Verbal Fluency Conditions 1 and 2, and the Wechsler Adult Intelligence Scale Fourth Edition Symbol Search and Coding Subsets; and the Cognitive Drug Research computerized test battery.

In some embodiments, biomarkers can be used for defining AD and for staging of the disease along its spectrum. Biomarkers of AD include but are not limited to ApoE isotype, amyloid PET, total Tau, phospho-Tau, pyroglutamate-Aβ, Aβ40, and Aβ42 in blood or CSF, and hippocampal volumetric (HCV) MRI.

Amyloid plaque burden in the brain can be measured by ¹⁸F-AV-45 PET. ¹⁸F-AV-45 is an amyloid ligand developed by Avid Radiopharmaceuticals (Philadelphia, Pennsylvania). It binds to fibrillar Aβ with a high affinity (Kd=3.1 nM). Results with 18F-AV-45 PET imaging have shown that patients with AD have selective retention of tracer in cortical areas expected to be high in amyloid deposition, whereas healthy controls have shown rapid washout from these areas, with only minimal cortical tracer retention. A significant difference in mean uptake of 18F-AV-45 has been observed between AD and age-matched control subjects. Test-retest variance of 18F-AV-45 PET imaging is low (less than 5%) in both AD patients and cognitively healthy controls. Visual interpretation of the 18F-AV-45 PET images and mean quantitative estimates of cortical uptake correlate with presence and quantity of amyloid pathology at autopsy as measured by immunohistochemistry and silver stain neuritic plaque score (Clark et al. 2011).

Morphometry MRI measures can also aid in the assessment of AD. These include whole brain volume, hippocampal volume, ventricle volume, and cortical gray matter volume. Cerebral blood flow as measured by ASL-MRI and functional connectivity as measured by tf-fMRI can be included in the assessment protocols. See, e.g., WO2016087944A2 for further description of PET imaging and other techniques associated with aiding in the assessment of AD. Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

Use of the anti-FSH antibody of the disclosure for the treatment of Alzheimer's disease results in an improvement in one or more of these parameters over baseline measurements or at least prevents or slows the progression of AD from one stage to the next stage.

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

The practice of the methods and compositions of the disclosure employs, unless otherwise indicated, conventional techniques of molecular biology (including recombinant techniques), cell culture, microbiology, cell biology, biochemistry, immunology, and neuroscience, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The following materials, reagents, and methods are used for the Examples described herein.

### Transgenic Mice

APP/PS1 mice, which were generated and bred at Mount Sinai, harbor a human transgene comprising APP^{K670N/M671L} and PSEN1^{ΔE9} (Minkeviciene, R. et al. J Neurochem 105, 584-594, (2008)). APP/PS1 mice develop amyloid plaques at 6 months, overt cognitive impairment at a later age of 12 months, and do not display neurofibrillary tangles Minkeviciene, R. et al. J Neurochem 105, 584-594, (2008); Onos, K. D. et al. PLOS Genet 15, (2019); Volianskis, A., et al. Neurobiol Aging 31, 1173-1187, (2010)). These mice, when bred on a pure C57BL/6J background, show evidence of sudden death due to seizure activity (Minkeviciene, R. et al. J Neurosci 29, 3453-3462, (2009)). Animal care and handling were performed according to NIH animal care guidelines at Mount Sinai. The protocols were reviewed and approved by the respective Institutional Animal Care and Use Committees.

### Safety Studies in Cynomolgus Monkeys

To understand the biodistribution and safety of the compositions disclosed herein as they may apply to humans, compositions comprising anti-FSH antibodies were injected as a single bolus into the tail veins of two male Cynomolgus monkeys aged 14 and 15 years, respectively. All monkeys studied (Macaca fascicularis), were fed Teklad Global 20% Protein Primate Diet. All protocols were approved by Institutional Animal Care and Use Committees of Icahn School of Medicine at Mount Sinai and Maine Medical Center Research Institute. The protocols were reviewed and approved by the respective Institutional Animal Care and Use Committees.

### Example 1: Anti-FSH Antibody Enhances Cognition in Ovariectomized AD Mice

To determine the effect of anti-FSH antibody on cognition in AD mice, 6.5-month-old female APP/PS1 mice on a C57BL/6J-congenic background are first ovariectomized and administered monoclonal anti-FSH Ab (Hu6) or human IgG (100 μg/mouse, daily, i.p.) for 4 weeks. Ovariectomy is known to induce uterine atrophy and elevate FSH levels (Sun L, et al., Cell. (2006) April 21; 125(2):247-60).

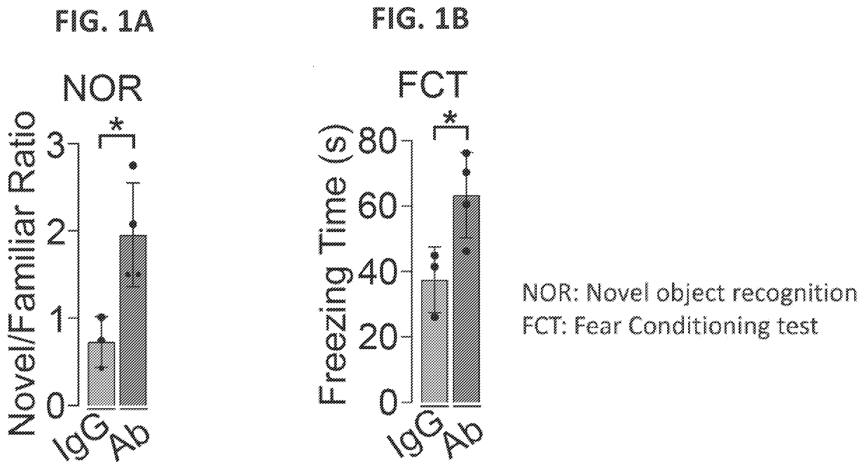

Cognition tests including the Novel Object Recognition (NOR) Test and Fear Conditioning Test (FCT) are then conducted. The NOR Test is a commonly utilized behavioral test, in which a mouse is presented with two identical objects during the first session, and then one of the two objects is replaced by a novel object during a second session (Leger, M. et al. Nat Protoc 8, 2531-2537 (2013)). On day 1, a habituation phase in an empty arena (for 5 minutes), is followed 24 hours later, by the training phase, which allows for a 5-minute exploration in the habituated arena where two identical objects are placed in opposite quadrants. The testing phase followed a gap of 20 minutes, which corresponds to a retention time frame that would normally allow memory deficits to be captured. For testing, one object is replaced with a novel object followed by 5 minutes of exploration. Parameters, including time spent, distance and number of head entries into novel/familiar quadrants, and object sniffing time, are collected using an ANY-maze video tracking system (Stoelting, UK). Mice receiving the anti-FSH antibody Hu6 show a higher ratio of time spent exploring the novel object relative to time exploring the familiar object (see FIG. 1A).

For FCT, mice receive context conditioning consisting of one foot shock (1.0 mA, 2 sec) per day for three days, with memory recall 24-h post-conditioning by measuring the time of freezing in context. Mice receiving the anti-FSH antibody Hu6 show improved cognition upon FSH blockade by Hu6 compared to IgG treated mice (see FIG. 1B). n=3-4 mice per group, p>0.05.

In a separate experiment, seven-week-old female APP/PS1 mice and wild type littermates were ovariectomized. After one week of recovery, mice were injected intraperitoneally with 100 μg Hu6 or human IgG for 4 months (5 days a week for the first two months, and 3 days a weeks for the next two months). Recognition and contextual memory in APP/PS1 mice, assayed using the Novel Object Recognition Test (FIG. 2A) and the Fear Conditioning Test (FIGS. 2B and 2C), respectively, were improved after 4 months of Hu6 injection. Statistics: Mean±SEM, two-way ANOVA, n=5-8 mice per group, *P<0.05, or as shown.

These results demonstrate that anti-FSH antibodies can improve cognition and memory in mice with Alzheimer's Disease.

### Example 2: Permeation of the Anti-FSH Antibody in the Mouse Brain

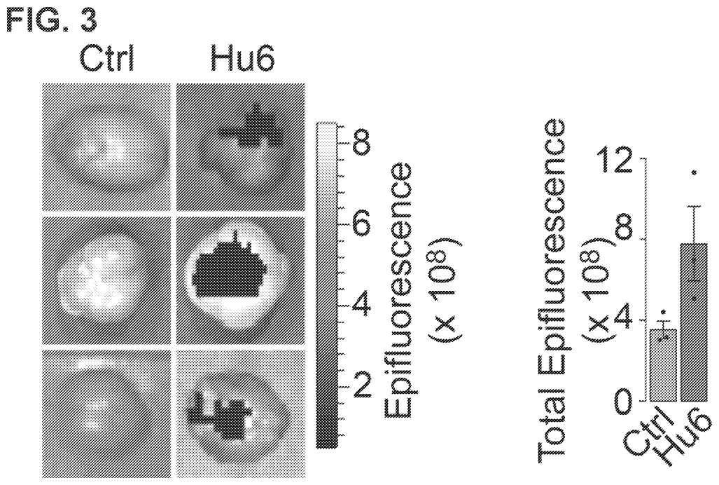

Alexa750-labeled humanized monoclonal FSH antibody, Hu6, was injected, i.p. into C57BL/6 mice. Epifluorescence (images and quantitation from three mice per group) on IVIS platform shows antibody localization in whole brain (post-perfusion) (FIG. 3). Control (Ctrl) is untagged Hu6.

### Example 3: Fine Mapping of the Anti-FSH Antibody

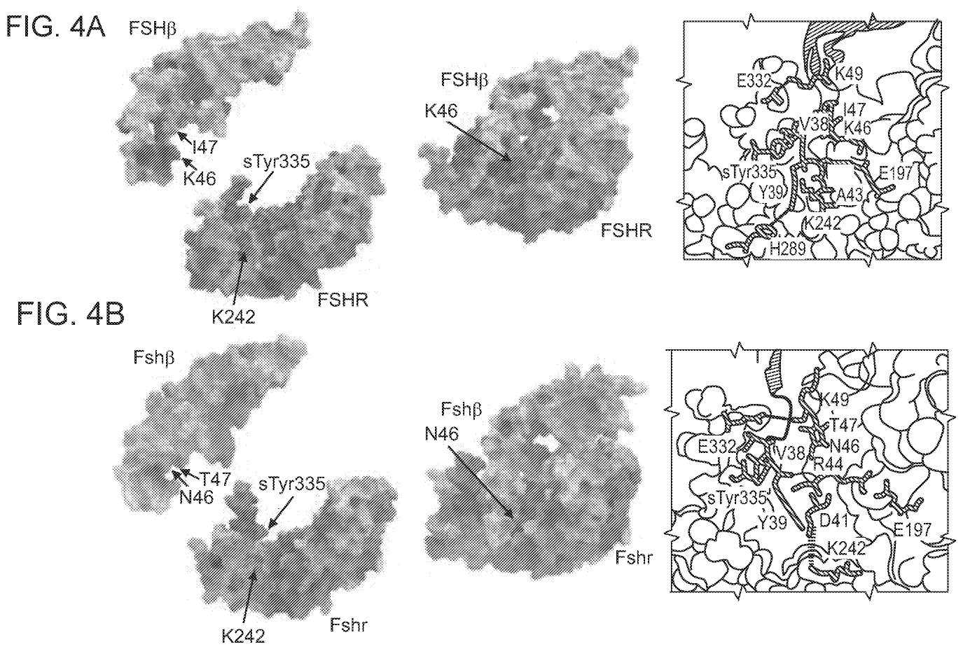

The atomic interactions between the anti-FSH antibody sequences (mouse monoclonal Hf2 antibody) and the respective parent FSH receptors (FSHR) were fine-mapped. The crystal structure of human FSH in complex with the entire ectodomain of the human FSHR (PDB ID 4AY9) was used as the template for comparative modeling (FIG. 4A). High sequence identity between mouse FSHR (UniProt id Q9QWV8) and human FSHR (88%) permitted for an accurate homology model to be constructed (FIG. 4B). In the human complex, residue R44 of β-chain forms an ion pair with residue E197 of the FSHR (FIG. 4A). Residue K49 forms another stable ion pair with residue E332 of the receptor. The sulphate group of the modified residue sTyr335 acts as hydrogen bond acceptor of the backbone amines of residues Y39 and V38. Furthermore, residue Y39 forms a hydrogen bond with residue H289 of the FSHR, and residue K242 is a hydrogen bond donor to the backbone of the residue A43. The modeled mouse FSHR-FSHB complex largely has a similar binding mode to the human complex, with an additional interaction through which residue D41 makes a hydrogen bond with residue K242 of the receptor (FIG. 4B). Of note is that the N46 and T47 residues in mouse FSHβ and corresponding K46 and I47 residues in human FSHB were found not to interact with the respective receptors (FIGS. 4A and 4B).

Without being bound by theory, it is believed that the example of this disclosure link FSH and AD, taken together with the strong clinical association of AD with rising serum FSH levels (Casadesus G, et al. J Biomed Biotechnol. (3):39508 (2006)), which provide the basis for the use of anti-FSH antibodies for the prevention and therapy of AD in women. Furthermore, as FSH levels also rise in aging men (Araujo, A. B. & Wittert, G. A. Clinical endocrinology & metabolism 25, 303-319, (2011), the exemplified finding that anti-FSH Ab improves cognition in mice enables the use of anti-FSH antibodies in both genders.

### Example 4: Safety of Anti-FSH Antibodies in a Cynomolgus Monkey Model

To understand the biodistribution and safety of the compositions disclosed herein as they may apply to humans, biodistribution and safety were evaluated in a Cynomolgus monkey model. After an overnight fast, ⁸⁹Zr-labelled anti-FSH antibodies were injected as a single bolus (1.3 mg, ˜1.3 mCi) into the tail veins of two male Cynomolgus monkeys aged 14 and 15 years, respectively. Blood was drawn via tail vein at five minutes, 30 minutes, and at 48 hours and 120 hour post-injection. Vitals, including mean arterial, systolic and diastolic blood pressure, respiratory rate, heart rate and rectal temperature, were recorded using Waveline Touch system (DRE) and Welch Allyn rectal thermometer.

Positron emission tomography (PET) and magnetic resonance (MR) images were acquired on a combined 3T PET/MRI system (Biograph mMR, Siemens Healthineers). Whole body MR images from each PET bed (head, thorax, pelvis) were automatically collated together with a scanner. MR parameters were as follows: acquisition plane, coronal; repetition time, 1000 ms; echo time, 79 ms; number of slices, 224; number of average, 2; spatial resolution of 0.6 mm×0.6 mm×1.0 mm; and acquisition duration, 29 min and 56 s per bed. After acquisition, PET raw data from each bed were reconstructed and collated offline using the Siemens proprietary e7tools with an ordered subset expectation maximization (OSEM) algorithm with point spread function (PSF) correction. A dual-compartment (soft tissue and air) attenuation map was used for attenuation. Image analysis was performed using Osirix MD, version 11.0. Whole-body MR images were fused with PET images and analyzed in an axial plane. Regions of interest (ROIs) were drawn on various tissues. The liver, kidney, BAT (interscapular region), subcutaneous WAT, visceral WAT, gonadal WAT, gallbladder, spleen, brain, and testes were traced in their entirety; bone marrow was imaged from the shoulder; and 3 lumbar vertebrae; and muscle was imaged from the quadriceps. Mean SUVs were calculated for each ROI. 89Zr-MS-Hu6 uptake of each tissue was expressed as the average of all mean SUV values per organ. Serum was collected for blood chemistry analysis by IDEXX BioAnalytics.

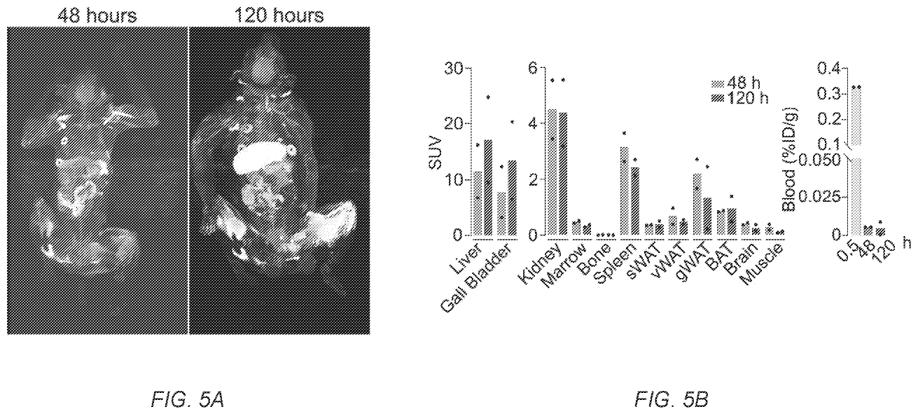

⁸⁹Zr-labelled anti-FSH antibodies peaked in the blood at five minutes, with a decline albeit with persistence in the serum at 48 hour and 120 hours (FIG. 5A). PET/CT scanning revealed high standard uptake values (SUV) in the liver and gall bladder, with lower SUVs in the kidney, spleen, fat depots, bone barrow, and the brain area (FIG. 5B).

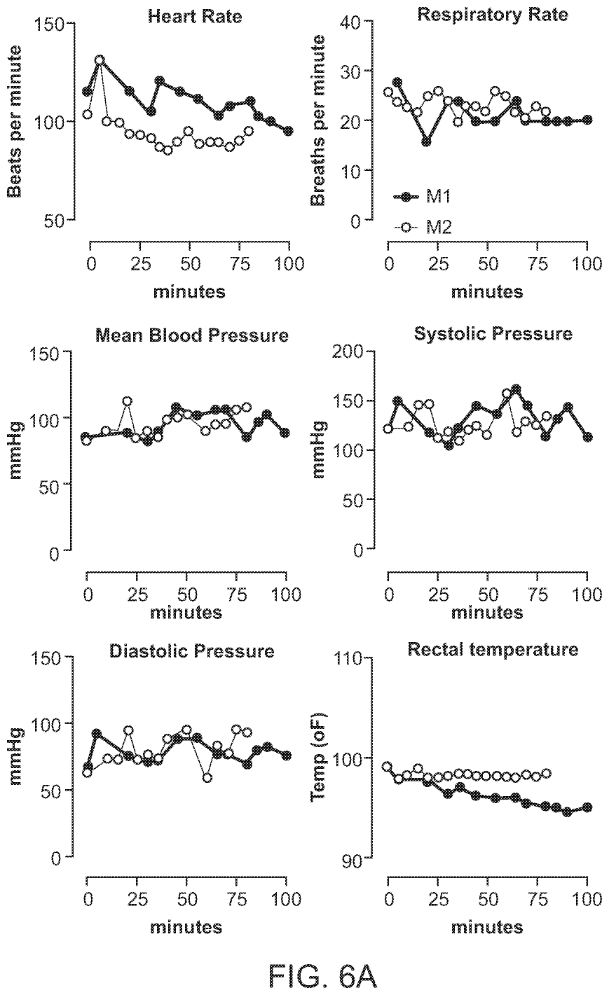

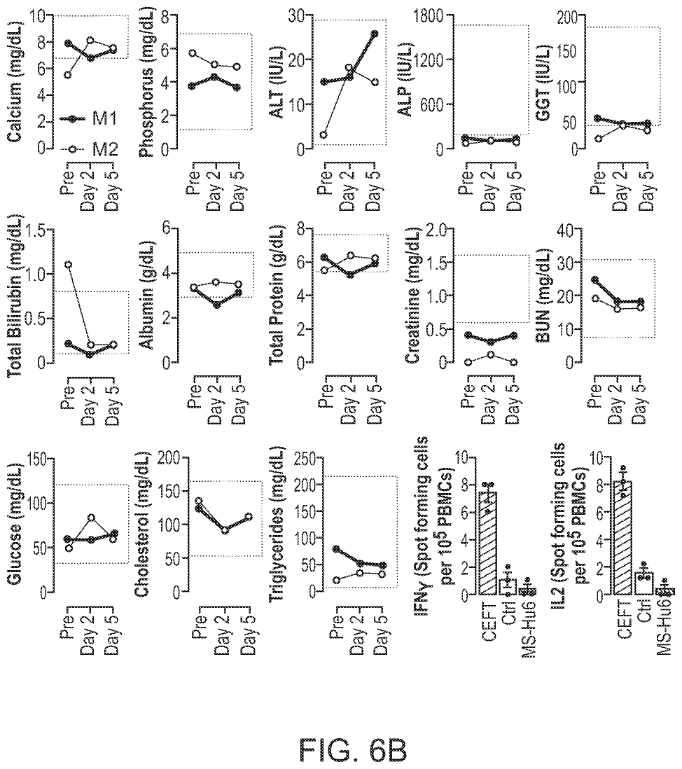

Standard safety parameters in treated monkeys were monitored up to 100 minutes, and no significant acute or delayed changes post-injection were observes for heart rate, respiratory rate, mean arterial blood pressure, systolic or diastolic blood pressure, or rectal temperature (FIG. 6A). Blood was also drawn at day 0 (pre-injection) and at days two and five post-injection. No concerning deviations from normative values were noted (FIG. 6B). These results indicate that at the dosage tested as a single intravenous bolus injection into monkeys, treatment with the composition comprising anti-FSH antibodies is generally safe.

### Example 5: Manufacturability and Formulation of Anti-FSH Antibody Compositions

Pharmaceutical compositions comprising antibodies selected on the basis of affinity, potency, specificity, functionality and pharmacokinetics can have physicochemical attributes making that affect manufacturing. It can therefore be useful to, at an early stage, determine physicochemical properties after a preliminary in silico screen of physiochemical properties. A computational tool, Protein-Sol, was used to screen anti-FSH compositions based on machine learning of amino acid sequences and physicochemical variables from 48 FDA-approved antibody compositions and 89 antibodies in late-stage clinical development (https://protein-sol.manchester.ac.uk/abpred). Protein-Sol uses antibody sequences (VH and VL) as inputs to provide predicted outputs for clone self-interaction by bio-layer interferometry (CSI-BLI), poly-specificity reagent (PSR), baculovirus particle ELISA (BVP-EL), cross-interaction chromatography (CIC), ELISA, accelerated stability (AS), hydrophobic interaction chromatography (HIC), stand-up monolayer adsorption chromatography (SMAC), salt gradient affinity capture (SGAC), expression titer in HEK cells (HEK), affinity capture (AC), and differential scanning fluorescence (DSF). It also provides a meta value for each sequence (1=best; 100=worst) by averaging ranks for 8 experimental parameters.

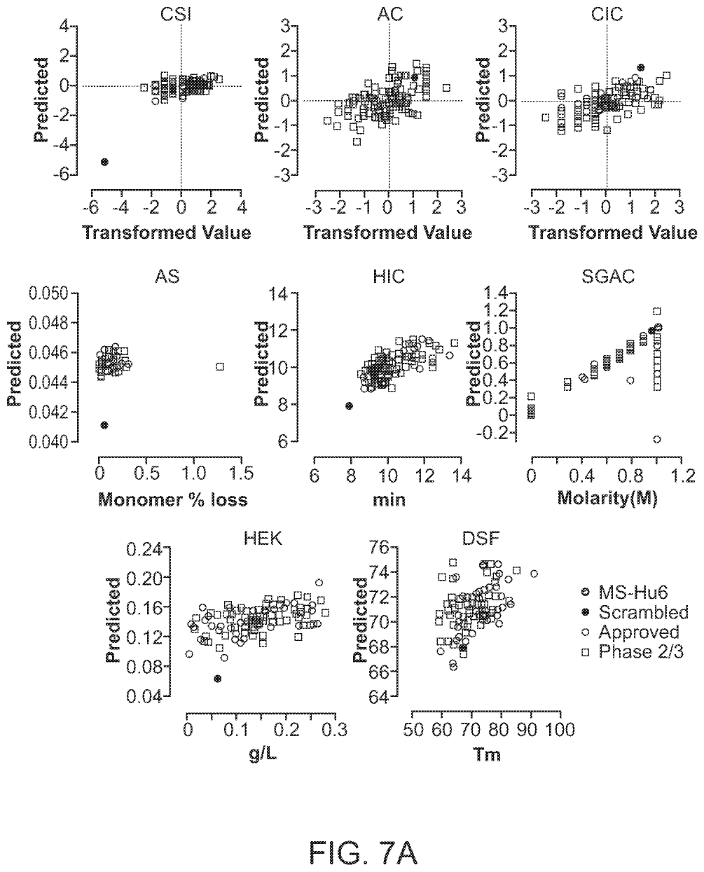

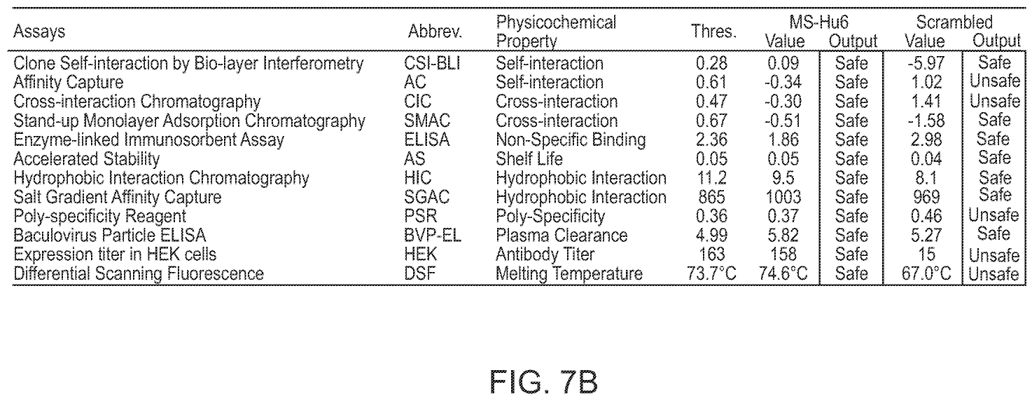

In the initial iteration, Protein-Sol provided predicted values for 12 separate physicochemical parameters that affect manufacturability. For all outputs, after inputting VH and VL regions, the anti-FSH compositions disclosed herein fell within acceptable thresholds, and were therefore deemed to be safe (FIG. 7A). These in silico results indicate the physicochemical properties of the anti-FSH antibody compositions disclosed herein were likely to have physiochemical parameters similar to FDA-approved antibodies.

For validation, a version of an anti-FSH antibody was produced wherein the CDR region was scrambled and 5 of 12 outputs, namely affinity capture, cross interaction chromatography (CIC), polyspecificity reagent (PSR), expression titers in HEK cells (HEK), and differential scanning fluorescence (DSF), fell outside the respective thresholds, an early indication that the scrambled version would present manufacturing challenges (FIG. 7B). In fact, while about 65% of FDA-approved monoclonal antibodies indicate no manufacturing challenges after such screening, those with some indications of manufacturability challenges have been FDA-approved. To complement data from individual outputs, a meta value was derived for the anti-FSH antibody composition disclosed herein and its scrambled sequence counterpart (1=best; 100=worst) by averaging ranks for 8 experimental parameters. We found that meta value pairs fell within the lower left quadrant, suggesting overall acceptable physicochemical properties, even in comparison with certain FDA-approved antibodies in the upper right quadrant (FIG. 7A).

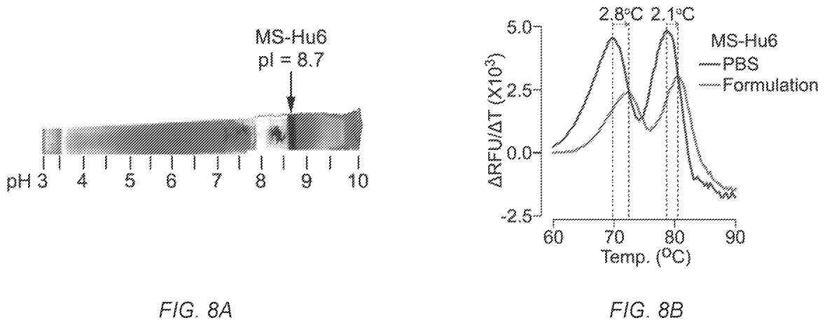

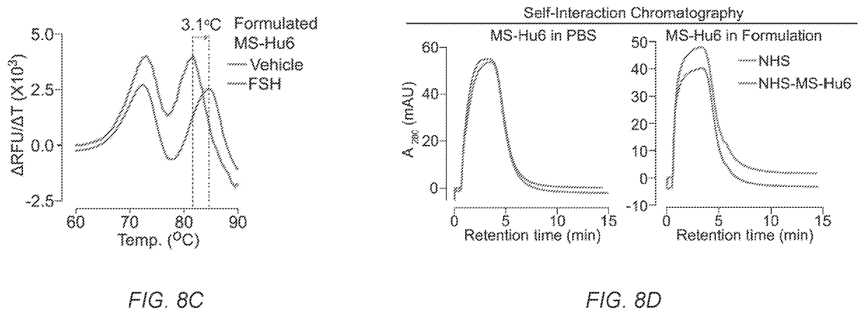

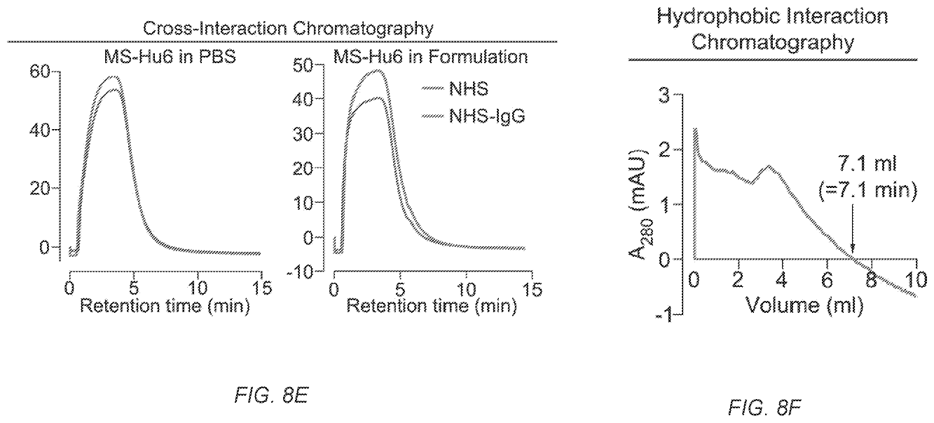

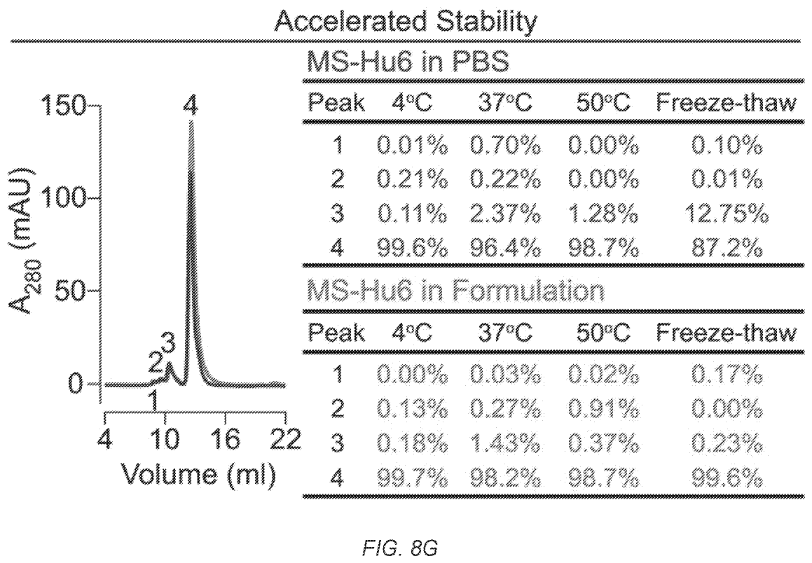

Before testing the physicochemical characteristics of the anti-FSH antibody composition experimentally, a preferred formulation was developed. To prevent deamidation and isomerization at neutral and basic pHs, therapeutic antibody compositions are typically formulated at pHs away from their isoelectric pH (pI). Using Expasy, the pI for the anti-FSH composition was predicted to be 8.58. Isoelectric focusing was used to determine the pI. Two-dimensional electrophoresis was performed by first rehydrating anti-FSH antibodies (500 μg) for 2 hours at room temperature in rehydration buffer (8M urea, 2% CHAPS, 0.5% IPG buffer, and trace of bromophenol blue) without DTT. The sample was then run on an 18 cm 3-10 strip using the Ettan IPGphor 3 Isoelectric Focusing System (GE Healthcare). Four voltage steps (50 V for 10 hours; 500 V for 1 hour; 1000 V for 1 hour; 8000V for 4 hours) were followed by Coomassie blue staining. Isoelectric focusing confirmed a pI pf 8.7 (FIG. 8A).

Next, 215 different anti-FSH antibody formulations comprising different combinations of salt, detergent and sugars were tested for thermal stability. The thermal shift assay used a fluorescent reporter, Sypro-Orange (Protein Thermal Shift Dye Kit, ThermoFisher, Catalog #4461146), to detect hydrophobic domains that are exposed following the heat-induced unfolding of globular proteins. anti-FSH antibodies (1.5 μg/μL), either in a test formulation or in PBS, were incubated with or without human FSH (0.5 μg/μL) at room temperature for 30 minutes, with fluorescence captured sequentially at 0.3° C. increments using a StepOne Plus Thermocycler (Applied Biosystems). Tm was calculated based on the inflection point of the melt curve, and thermal shift was derived from ΔTₘ=TₘA−TₘB.

This testing yielded an anti-FSH antibody formulation comprising a stock solution of 2 mg/mL in 20 mM phosphate, 0.001% (v/v) Tween-20, 1 mM NaCl, and 260 mM sucrose (pH=6.58). Both the Fc and Fab regions of the anti-FSH antibody formulation showed a thermal shift (ΔTₘ) compared with anti-FSH antibodies in PBS, indicating the anti-FSH antibody formulation as being more stable (FIG. 8B) than in PBS. The binding of formulated anti-FSH antibodies to purified human FSH was tested. A ΔTₘ of 3.1° C. of the Fab, and not the Fc region, established greater thermal stability due to FSH binding (FIG. 8C). Further, relative to anti-FSH antibodies in PBS, the formulated anti-FSH antibodies showed dampened peak signals (FIG. 8B). These results indicates that a lower number of antibody molecules underwent unfolding—indicating enhanced stability—despite identical added concentrations (20 μg/well).

In order to further characterize the formulated anti-FSH antibodies physicochemically, a series of biochemical tests, namely cross-interaction chromatography (CIC), self-interaction chromatography (SIC), size exclusion chromatography (SEC), hydrophobic chromatography, and polyspecificity assays were performed. For CIC and SIC, an in-house NHS ester column was developed, conjugated either with human IgG (for CIC) or MS-Hu6 (for SIC). anti-FSH antibodies were passed through an unconjugated column both in PBS and as the formulation described above, as well as through the two conjugated columns. As shown in FIG. 8D and FIG. 8E, retention times of anti-FSH antibodies in the respective conjugated columns were not different from those of the unconjugated column. These results indicate that the anti-FSH antibody does not appreciably interact with itself or with human IgGs, also indicative of little or no aggregation.

Next, a butyl sepharose column was used, and anti-FSH antibodies were passed through the column in 1.8 M NH₂SO₄ and 0.1 M Na₂PO₄ at pH 6.5 over 20 minutes at a flow rate of 1 mL/min. UV absorbance was monitored at 280 nm to yield a retention time of 7.1 minutes (FIG. 8F), which is below the theoretical threshold of 11.2 min (FIG. 7B).

Anti-FSH antibodies both in PBS and as the formulation described above were stressed through three freeze-thaw cycles (from −80° C. to room temperature) and by incubation for 1 week at 4° C., 37° C. or 50° C., followed, in all cases, by SEC (FIG. 8G). We noted a major peak (#4) and three minor high molecular weight peaks (#1 to 3). Areas under peaks 1 and 2 were between 0 and 0.91% of the total eluate under all conditions for anti-FSH antibodies both in PBS and as the formulation described above. Peak 3 remained generally low (<1.5%) with anti-FSH antibodies in the formulation, but was considerably higher (up to 12.7%) during freeze-thaw for anti-FSH antibodies in PBS. Furthermore, the major peak 4 was consistently >99% with anti-FSH antibodies in the formulation, particularly when compared to anti-FSH antibodies in PBS under freeze thaw (87.2%). Taken together, these results indicate that the formulation described above protected against aggregation even under extreme stress conditions. Extending the protocol to 25 mL elution yielded no fragment peaks under any condition (FIG. 8G).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating Alzheimer's Disease (AD), preventing the onset of AD, reducing cognitive or functional decline, or reducing symptom load in AD a subject in need or at risk thereof, comprising administering to said subject a therapeutically effective amount of an anti-Follicle Stimulating Hormone (FSH) antibody or an antigen-binding portion thereof, wherein the anti-FSH antibody, or antigen-binding portion thereof comprises
(a) a heavy chain variable sequence comprising a sequence that is at least 90% identical to a sequence selected from SEQ ID NOs: 3, 11, 13, and 15;
(b) a light chain variable sequence comprising a sequence that is at least 90% identical to a sequence selected from SEQ ID NOs: 4, 12, 14, and 17;
(c) a heavy chain CDR1 (CDRH1) comprising SEQ ID NO:5;
(d) a heavy chain CDR2 (CDRH2) comprising SEQ ID NO:6;
(e) a heavy chain CDR3 (CDRH3) comprising SEQ ID NO:7;
(f) a light chain CDR1 (CDRL1) comprising SEQ ID NO:8;
(g) a light chain CDR2 (CDRL2) comprising SEQ ID NO:9; and
(h) a light chain CDR3 (CDRL3) comprising SEQ ID NO:10.

2. The method of claim 1, wherein the subject has a condition in which FSH levels are elevated.

3-5. (canceled)

6. The method of claim 2, wherein the condition is a genetic disease, chemotherapy, surgical menopause, or orchiectomy.

7. The method of claim 6, wherein the genetic disease is Turners syndrome.

8. The method of claim 1, wherein the method alters one or more of the following in the subject in need thereof:
(a) reduces Aβ accumulation;
(b) reduces amyloid plaques;
(c) reduces Tau accumulation in the brain; and
(d) enhances cognitive function.

9. The method of claim 8, wherein the one or more of Aβ accumulation, amyloid plaques, and Tau accumulation in the brain is lower by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, as compared to the corresponding reference levels in the subject or in a control.

10. The method of claim 8, wherein the cognitive function is enhanced by at least about 20%, at least about 30%, at least about 40%, or at least about 50%, at least about 60%, at least about 70%, at least about 80%, or at least about 90%, as measured on one or more tests selected from the group consisting of the Alzheimer's Disease Assessment Scale-cognitive subscale (ADAS-cog); clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB);
the Sandoz Clinical Assessment-Geriatric (SCAG), the Buschke Selective Reminding Test; the Verbal Paired Associates subtest; the Logical Memory subtest: the Visual Reproduction subtest of the Wechsler Memory Scale-Revised (WMS-R); the explicit 3-alternative forced choice task;
and the Benton Visual Retention Test.

11. The method of claim 1, wherein the subject is concurrently treated with one or more agents selected from the group consisting of a cholinesterase inhibitor, an N-methyl-D-aspartate (NMDA) receptor antagonist, a hormone, a vitamin, an antipsychotic, a tricyclic antidepressant, a benzodiazepine, insulin, adeno-associated virus delivery of NGF, CERE-110, beta-blocker, human amyloid vaccine, beta or gamma secretase inhibitor, nicotinic or muscarinic agonist, and a second antibody.

12. The method of claim 11, wherein the cholinesterase inhibitor is selected from the group consisting of galantamine, rivastigmine, tacrine, and donepezil.

13. The method of claim 11, wherein the NMDA receptor antagonist is selected from the group consisting of ketamine, methadone, memantine, amantadine, and dextromethorphan or a salt thereof.

14. The method of claim 11, wherein the antipsychotic agent is selected from the group consisting of aripiprazole, risperidone, olanzapine, quetiapine, or haloperidol.

15. The method of claim 11, wherein the benzodiazepine is selected from the group consisting of lorazepam, oxazepam and temazepam.

16. The method of claim 11, wherein the tricyclic antidepressant is nortriptyline.

17. The method of claim 11, wherein the agent is a hormone selected from the group consisting of estrogen, progesterone and leuprolide.

18. The method of claim 11, wherein the agent is a vitamin selected from the group consisting of folate and nicotinamide.

19. The method of claim 11, wherein the second antibody is selected from the group consisting of bapineuzumab, solanezumab, gantenerumab, crenezumab, ponezumab, BAN2401, and aducanumab.

20-22. (canceled)

23. The method of claim 1, wherein the cognitive decline is assessed by determining the subject's score before and after administration of said anti-FSH antibody or antigen-binding fragment thereof, using an Alzheimer's Disease Assessment Scale-Cognition (ADAS-Cog) test.

24. The method of claim 23, wherein the reduction in cognitive decline as measured by ADAS-Cog is at least 10%, at least 15%, at least 20%, at least 25%, at least 30%, at least 35%, at least 40%, or at least 45% relative to a placebo.

25. (canceled)

26. The method of claim 1, wherein the treatment is prophylactic for completely or partially preventing AD or symptoms thereof in the subject, or the treatment is therapeutic for partially or completely curing AD or symptoms associated with AD in the subject.

27. (canceled)

28. A pharmaceutical composition comprising an isolated anti-Follicle Stimulating Hormone (FSH) antibody, or antigen-binding portion thereof, and a pharmaceutically acceptable carrier or excipient, wherein the composition is capable of crossing the blood brain barrier from the blood into the brain, wherein the anti-FSH antibody or antigen-binding portion thereof comprises
(a) a heavy chain variable sequence comprising a sequence that is at least 90% identical to a sequence selected from SEQ ID NOs: 3, 11, 13, and 15;
(b) a light chain variable sequence comprising a sequence that is at least 90% identical to a sequence selected from SEQ ID NOs: 4, 12, 14, and 17;
(c) a heavy chain CDR1 (CDRH1) comprising SEQ ID NO:5;
(d) a heavy chain CDR2 (CDRH2) comprising SEQ ID NO:6;
(e) a heavy chain CDR3 (CDRH3) comprising SEQ ID NO:7;
(f) a light chain CDR1 (CDRL1) comprising SEQ ID NO:8;
(g) a light chain CDR2 (CDRL2) comprising SEQ ID NO:9; and
(h) a light chain CDR3 (CDRL3) comprising SEQ ID NO:10.

29. (canceled)
